(19) United States
(12) Patent Application Publication
(10) Pub. No.: US 2026/0354715 A1
(43) Pub. Date: Nov. 12, 2026

(54) CONTINUOUS ANALYTE MONITORING DEVICE

(71) Applicant: Roche Diabetes Care GmbH, Mannheim (DE)

(72) Inventors: Olaf Roscher, Schriesheim (DE); Harald von Campenhausen, Ladenburg (DE)

(21) Appl. No.: 19/714,226

(22) Filed: Jun. 19, 2026

Related U.S. Application Data
(63) Continuation of application No. PCT/EP2024/086465, filed on Dec. 16, 2024.

(30) Foreign Application Priority Data
Dec. 20, 2023 (EP) 23 218 470.5

(51) Int. Cl. A61B 5/145; A61B 5/00

(52) U.S. Cl. CPC A61B 5/14546; A61B 5/0002; A61B 5/14503

(57) ABSTRACT

An analyte monitoring device has a sensor that detects an analyte in a body fluid. The sensor has an insertable portion that is inserted into the body tissue. An insertion component has an insertion cannula and cannula holder. The insertion cannula is attached to the holder and the sensor is at least partially placed inside the cannula. A removable sterility cap at least partially surrounds the insertable portion. The device has a housing that includes an open channel around the sensor and the insertion component, an electronics compartment, lower and upper housing portions, and a sealing channel with input and output filling ports. The sealing channel is filled with sealing material that seals an interior space enclosed by the housing portions. The removable sterility cap, open channel and insertion cannula holder form a sterile compartment for the insertion cannula and at least the insertable portion of the sensor.

### RELATED APPLICATIONS

This application is a continuation of PCT/EP2024/086465, filed Dec. 16, 2024, which claims priority to European Patent Application No. 23 218 470.5, filed Dec. 20, 2023, the entire disclosures of both of which are hereby incorporated herein by reference.

### BACKGROUND

This disclosure relates to a continuous analyte monitoring device, to a continuous analyte monitoring system, to a method of assembling a continuous analyte monitoring device and to a method of using a continuous analyte monitoring device. The devices and methods according to this disclosure may mainly be used for long-term monitoring of an analyte concertation in a body fluid, such as for long-term monitoring of a blood glucose level or of the analyte concentration of one or more other types of analytes in a body fluid. This disclosure may both be applied in the field of home care as well as in the field of professional care, such as in hospitals. Other applications are feasible.

Monitoring certain body functions, more particularly monitoring one or more concentrations of certain analytes, plays an important role in the prevention and treatment of various diseases. Without restricting further possible applications, this disclosure will be described in the following text with reference to blood-glucose monitoring. However, additionally or alternatively, this disclosure can also be applied to other types of analytes.

Blood glucose monitoring, besides by using optical measurements, specifically may be performed by using electrochemical biosensors. Examples of electrochemical biosensors for measuring glucose, specifically in blood or other body fluids, are known from U.S. Pat. No. 5,413,690 A, U.S. Pat. No. 5,762,770 A, U.S. Pat. No. 5,798,031 A, U.S. Pat. No. 6,129,823 A or U.S. Publication No. 2005/0013731A1 .

In addition to so-called spot measurements, in which a sample of a bodily fluid is taken from a user in a targeted fashion and examined with respect to the analyte concentration, continuous measurements are increasingly becoming established. Thus, in the recent past, continuous measuring of glucose in the interstitial tissue (also referred to as continuous monitoring, CM), for example, has been established as another important method for managing, monitoring and controlling a diabetes state.

In the process, an active sensor region is applied directly to a measurement site, which is generally arranged in the interstitial tissue, and, for example, converts glucose into electrical charge by using an enzyme (e.g., glucose oxidase, GOD), which charge is related to the glucose concentration and can be used as a measurement variable. Examples of such transcutaneous measurement systems are described in U.S. Pat. No. 6,360,888 B1 or in U.S. Publication No. 2008/0242962A1 .

Hence, current continuous monitoring systems typically are transcutaneous systems or subcutaneous systems, wherein both expressions, in the following, will be used equivalently. This means that an actual sensor or at least a measuring portion of the sensor may be arranged under a skin of the user. However, an evaluation and control part of the system (also referred to as a patch) may be generally situated outside of the body of the user, outside of an human or animal body. In the process, the sensor maybe generally applied using an insertion instrument, which is likewise described in U.S. Pat. No. 6,360,888 B1 in an exemplary fashion. Other types of insertion instruments are also known.

The sensor typically comprises a substrate, such as a flat substrate, onto which an electrically conductive pattern of electrodes, conductive traces and contact pads may be applied. In use, the conductive traces typically are isolated by using one or more electrically insulating materials. The electrically insulating material typically further also acts as a protection against humidity and other detrimental substances and, as an example, may comprise one or more cover layers such as resists.

Current continuous glucose monitoring devices may have a connector which comprises a vertical opening that is part of a sterility unit which houses a part of the sensor to be inserted into the skin and an insertion cannula. A holder of the insertion cannula may seal an upper side of the connector whereas a sterility cap may seal a lower side of the connector. The sensor may penetrate the connector horizontally to connect to an electronics unit and it is important for a preservation of a sterile state that a horizontal channel of the connector which envelops the sensor on its way to the electronics unit is tightly sealed. Sealing the sensor while passing through the connector is a technical challenge. Moreover, commonly, after the sensor has been mounted on a lower sensor patch plate, a sensor patch connector and on top of that a sealing element commonly needs to be mounted which renders the device complex.

Further, in current continuous glucose monitoring devices, a connection between housing parts is usually made using adhesive filled within grooves. Air bubbles can form in the adhesive when the adhesive is applied. A housing part may be fitted from above and air may be displaced and has to escape through a ventilation hole. If the housing part is not fitted exactly vertically when it comes into contact with the adhesive, air bubbles can form at contact points. A disadvantage of air bubbles in a bond may be that they represent a weak point in a watertight bond.

EP 3 727 130 B1 describes a medical system. The medical system comprises: a. a housing; b. a preassembled functional module received in the housing, the preassembled functional module comprising b1.an analytical sensor for detecting at least one analyte in a body fluid of a user; b2. an electronics unit electrically connected to the analytical sensor; and b3. an insertion component for inserting the analytical sensor into a body tissue of the user; c. at least one removable protective cap connected to the housing, covering the preassembled functional module.

EP 3 202 324 A1 describes a medical device for detecting at least one analyte in a body fluid, a method for assembling the medical device and a method of using the medical device. The medical device comprises at least one analyte sensor comprising an insertable portion adapted for at least partially being inserted into a body tissue of a user; at least one insertion cannula, wherein the analyte sensor at least partially is placed inside the insertion cannula; at least one electronics unit, wherein the analyte sensor is operably connected to the electronics; at least one housing, wherein the housing comprises at least one electronics compartment configured to at least partially receive the electronics unit and at least one sensor compartment configured to at least partially receive the analyte sensor. The sensor compartment forms a sealed compartment receiving at least the insertable portion of the analyte sensor. The sealed compartment comprises at least one detachable upper cap and at least one detachable lower cap. The detachable lower cap is configured for detachment before insertion, thereby opening the insertable portion for insertion. The insertion cannula is attached to the detachable upper cap. The detachable upper cap is configured for detachment after insertion, thereby removing the insertion cannula. The electronics compartment at least partially surrounds the sensor compartment.

EP 3 202 323 A1 describes a medical device for detecting at least one analyte in a body fluid. The medical device comprises: at least one analyte sensor comprising an insertable portion adapted for at least partially being inserted into a body tissue of a user; at least one insertion cannula, wherein the analyte sensor at least partially is placed inside the insertion cannula; at least one housing, wherein the housing comprises at least one sensor compartment, wherein the sensor compartment forms a sealed compartment receiving at least the insertable portion of the analyte sensor, wherein the sealed compartment comprises at least one detachable upper cap and at least one detachable lower cap, wherein the detachable lower cap is configured for detachment before insertion, thereby opening the insertable portion for insertion, wherein the insertion cannula is attached to the detachable upper cap, wherein the detachable upper cap is configured for detachment after insertion, thereby removing the insertion cannula; and at least one electronics unit, wherein the analyte sensor is operably connected to the electronics unit, wherein the electronics unit comprises at least one interconnect device with at least one electronic component attached thereto, wherein the interconnect device fully or partially surrounds the housing.

U.S. Publication No. 2022/0079475 A1 describes a system, apparatus, or device that includes an analyte sensor for monitoring analyte levels. The system, apparatus, or device can include a printed circuit board configured to monitor an analyte level, and a battery connected to the printed circuit board and configured to power the printed circuit board. The system, apparatus, or device can also include a connector connected to the printed circuit board and configured to establish an electrical connection between an analyte sensor and the printed circuit board, and a processor connected to the printed circuit board and configured to process data associated with the monitored analyte level. In addition, the system, apparatus, or device, can include an antenna for transmitting the monitored analyte level resting on a plurality of risers. The risers can extend from a surface of the printed circuit board by a fixed distance.

CN 218451759 U describes a sterilization module with a monitoring treatment probe, a medical instrument and an implanter. The medical instrument mainly comprises a sterilization module, the sterilization module further comprises a sterilization shell, at least part of the sterilization shell forms a sterilization cavity, at least one end of the sterilization cavity is provided with an opening, and the in-vivo part of the monitoring treatment probe and the part of the guide implant are placed in the sterilization cavity; the guide implant can fix and cover the opening of the sterilization cavity, and a seal is formed between the sterilization shell and the guide implant. The sterilization module disclosed by the utility model is small in size and convenient for sterilization operation, and the number of sterilization modules with the same size is increased, so that the sterilization cost is reduced. After being sterilized, the sterilization module is matched with other modules to form a medical instrument, the whole medical instrument is directly formed when a user uses the medical instrument, and the medical instrument is loaded on an implanter without additional assembly of the user; the implanter is designed with low cost and has a mistaken touch prevention function; users can use the product safely, conveniently and inexpensively.

### SUMMARY

This disclosure teaches a continuous analyte monitoring device, a continuous analyte monitoring system, a method of assembling a continuous analyte monitoring device and a method of using a continuous analyte monitoring device, which solve at least one of the problems mentioned above. In particular, this disclosure teaches a reliable sealing of the continuous analyte monitoring device.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “an element” and “the element” may indicate that several elements such as at least two of the elements may be present in particular embodiments.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “groove,” “housing,” “cavity,” and “sealing channel,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Further, the term “essentially parallel” may comprise slight deviations from a parallel arrangement such as arrangements which deviate from a parallel arrangement by no more than 10 degrees, preferably by no more than 5 degrees. The term “essentially perpendicular” may comprise slight deviations from a perpendicular arrangement such as arrangements which deviate from a perpendicular arrangement by no more than 10 degrees, preferably by no more than 5 degrees.

In a first aspect of this disclosure, a continuous analyte monitoring device is disclosed, comprising:
an analyte sensor comprising an insertable portion adapted for at least partially being inserted into a body tissue of a user, wherein the analyte sensor is configured for detecting an analyte in a body fluid of the user;
an insertion component comprising an insertion cannula and an insertion cannula holder, wherein the insertion cannula is attached to the insertion cannula holder, wherein the analyte sensor is at least partially placed inside the insertion cannula;
a removable sterility cap, wherein the removable sterility cap at least partially surrounds the insertable portion of the analyte sensor;
a housing, wherein the housing comprises an open channel which at least partially surrounds the analyte sensor and the insertion component, wherein the housing further comprises an electronics compartment with an electronics unit received therein; wherein the housing is at least partially formed by a lower housing portion and by an upper housing portion, wherein the housing comprises a sealing channel comprising an input filling port and an output filling port, wherein the sealing channel is at least partially filled with a sealing material being configured for sealing an interior space enclosed by the upper housing portion and the lower housing portion;
   wherein the removable sterility cap, the open channel of the housing and the insertion cannula holder from a sterile compartment for the insertion cannula and at least the insertable portion of the analyte sensor.

This disclosure may specifically be associated with at least one of the advantages described in the following: the subject matter of this disclosure may reduce a complexity in product design which in turn may reduce production costs compared to hitherto known continuous analyte monitoring devices. Specifically, this disclosure may improve a design of continuous analyte monitoring devices which employ a sterile compartment where one part of the analyte sensor forms part of the sterile compartment whereas another part of the analyte sensor leaves the sterile compartment to enter into an electronics compartment which comprises an electronics unit while maintaining an integrity of the sterile compartment and sealing it off from the electronics compartment and/or the environment, respectively. This in turn may also simplify sterilization, sealing and assembly of the embodiments of this disclosure.

The term “user” as used herein is a broad term and is to be given its ordinary and customary meaning to a person of ordinary skill in the art and is not to be limited to a special or customized meaning. The term exemplarily relates to a person intending to monitor an analyte value, such as a glucose value, in a person's body tissue. In an embodiment, the term specifically may refer, without limitation, to a person using the continuous analyte monitoring device which will further be described below in more detail. For example, the user may be a patient suffering from a disease, such as diabetes. The user may also be referred to as subject or as patient. However, in another embodiment, the person using the continuous analyte monitoring device is different from the user.

The term “continuous analyte monitoring device” as used herein is a broad term and is to be given its ordinary and customary meaning to a person of ordinary skill in the art and is not to be limited to a special or customized meaning. The term specifically relates to a medical device which is configured for use in the field of medical technology, exemplarily in the field of medical analytics or medical diagnostics. The continuous analyte monitoring device may be configured for performing a medical function and/or for being used in a medical process, such as in one or more of a therapeutic process, a diagnostic process or another medical process.

The continuous analyte monitoring device specifically may comprise an assembly of two or more components capable of interacting with each other, such as in order to perform one or more diagnostic and/or therapeutic purposes, such as in order to perform a medical analysis. Specifically, the two or more components may be capable of performing a detection of the analyte in the body fluid and/or of contributing to the detection of the analyte in the body fluid. The continuous analyte monitoring device generally may also be referred to as a sensor assembly, a sensor system, a sensor kit, a sensor device or a medical device.

The continuous analyte monitoring device specifically may be configured for monitoring or detecting a presence of the analyte in the body tissue and/or in the body fluid and/or may be configured for monitoring or detecting a concentration of the analyte in the body tissue and/or in the body fluid, specifically over time or in a time-dependent manner. Specifically, the continuous analyte monitoring device may be configured for acquiring and evaluating a data stream of time-dependent concentrations of the analyte. The data stream may be a continuous data stream. However, the data stream may comprise or may have one or more gaps wherein, during the gaps, no data may be acquired. Further, a number of data elements or signals per time unit which are acquired may vary over time. Specifically, the continuous analyte monitoring device may be configured for comparing a value of a concentration of the analyte with one or more threshold values. Further, specifically, the continuous analyte monitoring device may be configured for outputting warning signals under certain circumstances.

Specifically, the continuous analyte monitoring device may be a continuous glucose monitoring device. A concentration of glucose in blood or body fluid of the user or the patient may be dependent on events which increase or decrease a concentration of glucose such as an intake of food or physical activity. Thus, the concentration of glucose in the blood or body fluid may be describable as time-dependent concentration, e.g., the concentration may vary or change over time. Thus, when evaluating the concentration at a first point in time, the concentration may have a first value and when evaluating the concentration at a second point in time, the concentration may have a second value which may be differ from the first value. The second value may be higher or lower than the first value. However, in certain scenarios, the first value may be equivalent to the second value.

The continuous analyte monitoring device may be configured to be mounted on a skin site of a body part, specifically of the user, selected from the group consisting of: an arm, exemplarily an upper arm; a stomach; a shoulder; a back; hip; and/or a leg. Specifically, the body part may be the upper arm. However, also other applications may be feasible.

The continuous analyte monitoring device may comprise a component which may be configured to stay outside of the body tissue. The component which may be configured to stay outside of the body tissue may specifically be the housing comprising the electronics compartment with the electronics unit received therein. Further, the analyte sensor, comprises, as outlined above, the insertable portion. The insertable portion may be configured for being inserted into the body tissue of the user.

The continuous analyte monitoring device may from a preassembled single unit. The term “preassembled” as used herein is a broad term and is to be given its ordinary and customary meaning to a per-son of ordinary skill in the art and is not to be limited to a special or customized meaning. The term specifically may refer, without limitation, to the fact that an assembly process has already taken place. Thus, the components of the continuous analyte monitoring device may already be assembled, such as by being mechanically and/or electrically interconnected, thereby being ready for use for the function, such as the medical function, e.g., for the analytical function. The preassembling specifically may take place in a factory, thereby rendering the continuous analyte monitoring device a factory-assembled functional module.

The term “body fluid” as used herein is a broad term and is to be given its ordinary and customary meaning to a person of ordinary skill in the art and is not to be limited to a special or customized meaning. The term specifically relates to a fluid which typically is present in a body or body tissue of a user or a patient and/or which may be produced by the body of the user or the patient. As an example for body tissue, interstitial tissue may be named. Thus, as an example, the body fluid may be selected from the group consisting of blood and interstitial fluid. However, additionally or alternatively, one or more other types of body fluids may be used, such as saliva, tear fluid, urine or other body fluids. During detection of the analyte, the body fluid may be present within the body or body tissue. Thus, specifically, as will be outlined in further detail below, the analyte sensor may be configured for detecting the analyte in the body tissue.

The term “analyte” as used herein is a broad term and is to be given its ordinary and customary meaning to a person of ordinary skill in the art and is not to be limited to a special or customized meaning. The term specifically relates to an arbitrary element, component or compound which may be present in a body fluid and a presence and/or a concentration of which may be of interest for a user, a patient or medical staff such as for a medical doctor. Particularly, the analyte may be or may comprise an arbitrary chemical substance or chemical compound which may take part in a metabolism of the user or the patient, such as a metabolite. As an example, the analyte may be selected from the group consisting of glucose, cholesterol, triglycerides, and/or lactate. Additionally or alternatively, however, other types of analytes may be used and/or any combination of analytes may be determined. However, specifically, the analyte may be glucose. In the following, the continuous analyte monitoring device may specifically be described with respect to glucose monitoring. The detection of the analyte specifically may be an analyte-specific detection.

The term “detecting” as used herein is a broad term and is to be given its ordinary and customary meaning to a person of ordinary skill in the art and is not to be limited to a special or customized meaning. The term specifically relates to a process of determining a presence and/or a quantity and/or a concentration of an analyte. Thus, the detection may be or may comprise a qualitative detection, simply determining the presence of the analyte or the absence of the analyte, and/or may be or may comprise a quantitative detection, which determines the quantity and/or the concentration of the analyte. As a result of the detection, a signal may be produced which characterizes an outcome of the detection, such as at least one measurement signal. The measurement signal specifically may be or may comprise an electronic signal such as a voltage and/or a current. The measurement signal may be or may comprise an analogue signal and/or may be or may comprise a digital signal.

The term “analyte sensor” as used herein is a broad term and is to be given its ordinary and customary meaning to a person of ordinary skill in the art and is not to be limited to a special or customized meaning. The term specifically relates to a sensor which is capable of qualitatively or quantitatively detecting a presence and/or a concentration of an analyte.

The analyte sensor may particularly be a transcutaneous sensor. The term “transcutaneous sensor” as used herein is a broad term and is to be given its ordinary and customary meaning to a person of ordinary skill in the art and is not to be limited to a special or customized meaning. The term specifically may refer, without limitation, to an arbitrary sensor which is adapted to be fully or at least partly arranged within a body tissue of a patient or a user. For this purpose, the analyte sensor comprises the insertable portion. The term “insertable portion” as used herein is a broad term and is to be given its ordinary and customary meaning to a person of ordinary skill in the art and is not to be limited to a special or customized meaning. The term specifically may refer, without limitation, to a part or component of an element configured to be insertable into an arbitrary body tissue. In order to further render the analyte sensor to be usable as a transcutaneous sensor, the analyte sensor may fully or partially provide a biocompatible surface, i.e., a surface which, at least during durations of use, do not have any detrimental effects on the user, the patient or the body tissue. Specifically, the insertable portion of the analyte sensor may have a biocompatible surface. As an example, the transcutaneous sensor, specifically the insertable portion, may fully or partially be covered with a biocompatible membrane, such as a polymer membrane or gel membrane which is permeable for the analyte and/or the body fluid and which, on the other hand, retains sensor substances such as one or more analyte detection agents within the sensor and prevents a migration of these substances into the body tissue. Other parts or components of the analyte sensor may stay outside of the body tissue. The other parts may be connectable to an evaluation device such as to the electronics units as will further be described below.

The transcutaneous sensor generally may be dimensioned such that a transcutaneous insertion is feasible, such as by providing a width in a direction perpendicular to an insertion direction of no more than 5 mm, preferably of no more than 2 mm, more preferably of no more than 1.5 mm. The sensor may have a length of less than 50 mm, such as a length of 30 mm or less, e.g., a length of 5 mm to 30 mm. As used herein, the term “length” may refer to a direction parallel to an insertion direction. It shall be noted, however, that other dimensions are feasible.

The analyte sensor may specifically be an electrochemical analyte sensor. The term “electrochemical sensor” as used herein is a broad term and is to be given its ordinary and customary meaning to a person of ordinary skill in the art and is not to be limited to a special or customized meaning. The term specifically may refer, without limitation, to a sensor which is configured to conduct an electrochemical measurement, specifically in order to detect an analyte in a body fluid of a user. The term “electrochemical measurement” may refer to a detection of an electrochemically detectable property of the analyte, such as to an electrochemical detection reaction. Thus, for example, the electrochemical detection reaction may be detected by comparing one or more electrode potentials. The electrochemical sensor specifically may be adapted to and/or may be usable to generate an electrical sensor signal which directly or indirectly indicates the presence and/or the extent of the electrochemical detection reaction, such as a current and/or a voltage. The detection may be analyte-specific. The measurement may be a qualitative and/or a quantitative measurement. Still, other embodiments are feasible.

The analyte sensor may comprise at least two electrodes. The term “electrode” as used herein is a broad term and is to be given its ordinary and customary meaning to a person of ordinary skill in the art and is not to be limited to a special or customized meaning. The term specifically may refer, without limitation, to an arbitrary element which is configured to or which is usable to electrically or electrochemically detect an analyte. Specifically, each electrode may comprise a conductive pad or conductive element, such as a metal pad and/or a metal element and/or a pad or element made of a conductive inorganic or organic material such as carbon and/or a conductive polymer. The conductive pad or conductive element may be uncovered and/or may be covered with an additional material, such as a sensor chemical. The at least two electrodes of the analyte sensor may be embodied such that an electrochemical reaction may take place at one or more of the electrodes, such as one or more working electrodes. Thus, the electrodes may be embodied such that an oxidation reaction and/or reduction reaction may take place at one or more of the electrodes. The electrochemical detection reaction may be detected by comparing one or more electrode potentials, such as an electrostatic potential of a working electrode with an electrostatic potential of one or more further electrodes such as a counter electrode or a reference electrode. Generally, the two or more electrodes may be used for one or more of an amperometric, an amperostatic, a potentiometric or a potentiostatic measurement. These types of measurements generally are known to the skilled person in the art of analyte detection, such as from WO 2007/071562 A1 and/or the prior art documents disclosed therein. For potential setups of the electrodes, electrode materials or measurement setups, reference may be made to this document. It shall be noted, however, that other setups, electrode materials or measurement setups may be used within this disclosure. The electrodes generally comprise an electrode conductor path configured for transmitting a sensor current for detecting the analyte. The electrode conductor path in turn may in particular embodiments be connected to the sensor electronics, such as via an electrode contact which connects with a corresponding contact of an electronic component of the sensor electronics.

The at least two electrodes may be a working electrode configured for detecting the analyte and a further electrode. The further electrode may be selected from the group consisting of: a counter electrode, a reference electrode, and a combined counter-reference electrode. Exemplarily, the analyte sensor may comprise a two-electrode sensor. The two-electrode sensor may comprise precisely two electrodes, such as a working electrode and a further electrode such as a counter electrode, e.g., a working electrode and a combined counter/reference electrode. The term “working electrode” as used herein is a broad term and is to be given its ordinary and customary meaning to a person of ordinary skill in the art and is not to be limited to a special or customized meaning. The term specifically may refer, without limitation, to an electrode being adapted for or being usable for performing an electrochemical detection reaction for detecting an analyte in a body fluid. The working electrode may have an analyte detection agent being sensitive to the analyte to be detected. The working electrode may further comprise a conductive working electrode pad. The conductive working electrode pad may be in contact with the analyte detection agent. Thus, the analyte detection agent may be coated onto the conductive working electrode pad. The analyte detection agent may form an analyte detection agent surface which may be in contact with the body fluid. As an example, the analyte detection agent surface may be an open analyte detection agent surface or may be covered by the above-mentioned membrane which is permeable to the analyte to be detected and/or to the body fluid or a part thereof, such that the analyte may interact with the analyte detection agent. For potential analyte detection agents and/or materials for the conductive working electrode pad, again, reference may be made to WO 2007/071562 A1 and/or the prior art documents disclosed therein. Other embodiments, however, are feasible. The one or more “working electrode pads” specifically may be formed by a dot, line or grid which each can form a coherent area of an electrode material. If more than one dot, line or grid of the electrode material is superimposed, the sensor may provide more than one electrode pad. All electrode pads together may build the working electrode. The sensor may comprise the working electrode with a number of electrode pads in a range from 1 to 50, preferably from 2 to 30, preferably from 5 to 20 electrode pads.

The term “analyte detection agent” as used herein is a broad term and is to be given its ordinary and customary meaning to a person of ordinary skill in the art and is not to be limited to a special or customized meaning. The term specifically may refer, without limitation, to an arbitrary material or a composition of materials adapted to change a detectable property in a presence of an analyte. This property may be an electrochemically detectable property. Specifically, the analyte detection agent may be a highly selective analyte detection agent, which only changes the property if the analyte is present in the body fluid whereas no change occurs if the analyte is not present. The degree or change of the property is dependent on the concentration of the analyte in the body fluid, in order to allow a quantitative detection of the analyte. As an example, the analyte detection agent may comprise an enzyme, such as glucose oxidase and/or glucose dehydrogenase.

The at least two electrodes may further comprise the counter electrode. The term “counter electrode” as used herein is a broad term and is to be given its ordinary and customary meaning to a person of ordinary skill in the art and is not to be limited to a special or customized meaning. The term specifically may refer, without limitation, to an electrode adapted for performing an electrochemical counter reaction and adapted for balancing a current flow required by a detection reaction at a working electrode. Additionally or alternatively the at least two electrodes may further comprise reference electrode. The reference electrode may have a stable and well-known electrode potential. The electrode potential may preferably be highly stable. The counter electrode and the reference electrode may be one of a common electrode or two separate electrodes. Again, for potential materials usable for the counter electrode and/or the reference electrode, reference may be made to WO 2007/071562 A1 and/or the prior art documents disclosed therein. Other embodiments, however, are feasible.

The electrodes, particularly the working electrode, the counter electrode and/or the reference electrode, may have an identical dimension. The term “dimension” may refer to one or more of a width, a length, a surface area, a shape of the working electrode, the counter electrode and/or the reference electrode. A shape of the electrodes may be determined by a manufacturing process, such as a cutting and/or a printing process. The shape may be rectangular or round. Still, other embodiments are feasible, such as embodiments in which the dimensions of the working electrode and the counter/reference electrodes differ and/or embodiments in which a non-circular shape or a non-rectangular shape is used. The electrodes may be made of a non-corrosive and non-passivating material. With regard to possible electrode materials, reference may be made to the prior art documents cited above.

The analyte sensor may comprise a carrier, specifically a substrate. The at least two electrodes, specifically the at least two electrode conductor paths may be disposed on the carrier. The term “carrier” as used herein is a broad term and is to be given its ordinary and customary meaning to a person of ordinary skill in the art and is not to be limited to a special or customized meaning. The term specifically may refer, without limitation, to an arbitrary element which is suitable to carry one or more other elements disposed thereon or therein. The term “substrate” as used herein is a broad term and is to be given its ordinary and customary meaning to a person of ordinary skill in the art and is not to be limited to a special or customized meaning. The term specifically may refer, without limitation, to an arbitrary flat element which has a lateral extension exceeding its thickness by at least a factor of 2, at least a factor of 5, at least a factor of 10, or even at least a factor of 20 or more.

The carrier, specifically the substrate, specifically may have an elongated shape, such as a strip-shape and/or a bar-shape. The substrate, as an example, may comprise a shaft, specifically a shaft having an elongate shape. For example, the shaft may have a shape selected from the group consisting of a strip, a needle, a tape. Also other shapes may be feasible.

The carrier, specifically the substrate, may be a flexible carrier or substrate, i.e., a carrier or substrate which may be bent or deformed by forces which usually occur during wearing and insertion into the body tissue, such as forces of 10 N or less. Specifically the carrier or the substrate may be made of or may comprise a deformable material, such as a plastic or malleable material and/or an elastic material. As an example, the carrier or the substrate may be or may comprise a foil, such as a foil made of one or more of a paper material, a cardboard material, a plastic material, a metal material, a ceramic material or a glass material. As an example, the carrier or the substrate may comprise a polyimide foil. The carrier or the substrate specifically may comprise an electrically insulating material, such as an electrically insulating plastic foil.

Specifically, the analyte sensor may be a needle-shaped or a strip-shaped analyte sensor with a flexible substrate and the electrodes disposed thereon. As an example, the analyte sensor may have a total length of 5 mm to 50 mm, e.g., a total length of 7 mm to 30 mm. The term “total length” within the context of this disclosure relates to the overall length of the analyte sensor which means a portion of the analyte sensor which is inserted and the portion of the analyte sensor which may stay outside of the body tissue. The portion of the analyte sensor which is inserted may also be called the in-vivo portion, the portion of the analyte sensor which may stay outside of the body tissue may also be called the ex vivo portion. For example, the in vivo portion may have a length in the range from 3 mm to 12 mm. The analyte sensor may further comprise a biocompatible cover, such as a biocompatible membrane which fully or partially covers the analyte sensor and which prevents the analyte detection agent from migrating into the body tissue and which allows for a diffusion of the body fluid and/or the analyte to the electrodes.

The term “insertion component” as used herein is a broad term and is to be given its ordinary and customary meaning to a person of ordinary skill in the art and is not to be limited to a special or customized meaning. The term specifically relates to an arbitrary element which may be insertable at least partially into the body tissue, particularly in order to deliver or to transfer a further element. The insertion component may be configured for supporting the insertion of the analyte sensor or the insertion of a part of the analyte sensor.

As outlined above, the insertion component comprises the insertion cannula. The term “insertion cannula” as used herein is a broad term and is to be given its ordinary and customary meaning to a person of ordinary skill in the art and is not to be limited to a special or customized meaning. The term specifically may refer, without limitation, to a hollow needle which may be at least partially or completely slotted. The analyte sensor may be received within the insertion cannula, such as within a lumen of the insertion cannula. The insertion cannula may comprise a tip or a sharp end for inserting the analyte sensor at least partially into the body tissue. The insertion cannula e.g., may comprise at least one cross-section selected from the group consisting of: round, elliptical, U shaped, V shaped. Still, other embodiments are feasible. Specifically, the insertion cannula may be a slotted cannula. Alternatively, the insertion cannula may be a non-slotted cannula. The insertion cannula may be configured to be inserted vertically or at an angle of 90° to 30° relative to the body tissue of the user.

As further outlined above, the insertion component further comprises the insertion cannula holder for the insertion cannula. The term “insertion cannula holder” as used herein is a broad term and is to be given its ordinary and customary meaning to a person of ordinary skill in the art and is not to be limited to a special or customized meaning. The term specifically relates to an arbitrary element which may be configured for holding an insertion cannula. The insertion cannula may be attached to the insertion cannula holder. Specifically, the insertion cannula may be fixedly attached to the insertion cannula holder. The insertion cannula holder may at least partially surround the insertion cannula. Specifically, the insertion cannula may have a first end and an opposing second end. The first end may have a tip or a sharp end for inserting the analyte sensor at least partially into the body tissue. The second end may be attached to the insertion cannula holder. Further details on the insertion cannula holder are given below.

As outlined above, the continuous analyte monitoring device further comprises the removable sterility cap.

The term “cap” as used herein is a broad term and is to be given its ordinary and customary meaning to a person of ordinary skill in the art and is not to be limited to a special or customized meaning. The term specifically relates to an arbitrary element which is configured to close or to seal a volume. Specifically, the cap may close or seal an opening of an arbitrary container. The “cap” may have the form of a half-shell, a hemisphere, an open container, a lid or a cover. Also other forms may be feasible.

The term “removable” as used herein is a broad term and is to be given its ordinary and customary meaning to a person of ordinary skill in the art and is not to be limited to a special or customized meaning. The term specifically relates to a property of an element of being removable from an arbitrary object. Thereby, a close bonding or contact or a connection between the element and the object may be disconnected. Generally, the element may be removable in a reversible manner wherein the element may be attachable and detachable from the object in a reversible manner or in an irreversible manner wherein the element may not be attachable to the object after detachment. Further details are given below.

The term “sterility cap” as used herein is a broad term and is to be given its ordinary and customary meaning to a person of ordinary skill in the art and is not to be limited to a special or customized meaning. The term specifically may refer, without limitation, to an element such as a cover which is configured for maintaining a sterile atmosphere in a space fully or partially surrounded by the element. The sterility cap, as an example, may be a rigid sterility cap, e.g., made of a rigid plastic material and/or a metal. The sterility cap, as an example, may have a rotational symmetry about an axis which, as an example, may be identical to a rotational symmetry axis of the protective cap and/or of a rotational symmetry axis of the housing. The sterility cap, as an example, may have an elongated shape, with a length exceeding its diameter or equivalent diameter by at least a factor of 2, more preferably by at least a factor of five. The sterility cap, as an example, may have a length of 5 to 20 mm, e.g., a length of 10 to 15 mm.

As outlined above, the removable sterility cap at least partially surrounds the insertable portion of the analyte sensor. Thus, the insertable portion may be at least partially received in the removable sterility cap. The removable sterility cap may be configured for removal before insertion of the insertable portion of the analyte sensor into the body tissue.

As outlined above, the continuous analyte monitoring device further comprises the housing. The term “housing” as used herein is a broad term and is to be given its ordinary and customary meaning to a person of ordinary skill in the art and is not to be limited to a special or customized meaning. The term specifically relates to an arbitrary element which is adapted to fully or partially surround and/or receive one or more elements in order to provide one or more of a mechanical protection, a mechanical stability, an environmental protection against moisture and/or ambient atmosphere, a shielding against electromagnetic influences or the like. Thus, the housing may simply provide a basis for attachment and/or holding one or more further components or elements. Additionally or alternatively, the housing may provide one or more interior spaces for receiving one or more further components or elements. The housing may specifically be manufactured by injection molding. However, other embodiments are feasible.

The housing may comprise an upper side and a lower side. The terms “upper side” and “lower side” may refer to two opposing sides of the housing. The terms “upper side” and “lower side” may be considered as description without specifying an order and without excluding a possibility that several kinds of upper sides and lower sides may be applied.

Specifically, the upper side may refer to a distal side of the housing. The term “distal side” as used herein is a broad term and is to be given its ordinary and customary meaning to a person of ordinary skill in the art and is not to be limited to a special or customized meaning. The term specifically may refer, without limitation, to an indication of a position of the side of the housing in relation to a user which is furthermost away from a skin site of the user. Exemplarily, for inserting the analyte sensor, the housing may be brought into contact with the skin site of the user. The distal side may refer to a side being distanced to the skin site of the user.

Specifically, the lower side may refer to a proximal side of the housing. The term “proximal side” as used herein is a broad term and is to be given its ordinary and customary meaning to a person of ordinary skill in the art and is not to be limited to a special or customized meaning. The term specifically may refer, without limitation, to an indication of a position of the side of the housing in relation to a user which is closest to a skin site of the user. Exemplarily, for inserting the analyte sensor, the housing may be brought into contact with the skin site of the user. The proximal side may refer to a side being in close proximity to or even in direct contact with to the skin site of the user.

As outlined above, the housing comprises the open channel. The housing may specifically comprise the upper side and the lower side. The open channel may connect the upper side and the lower side. The housing may specifically be at least partially formed as a cylindrical ring surrounding the open channel.

The term “channel” as used herein is a broad term and is to be given its ordinary and customary meaning to a person of ordinary skill in the art and is not to be limited to a special or customized meaning. The term specifically relates to an arbitrary element which may have an elongate shape and which may provide a free volume or lumen and which enables other elements to pass there through. The channel may specifically be an essentially straight, curved or circular shaped channel. As further used herein, the term “straight” may refer to a continuous extension of the channel in one direction essentially without a bend, angle or curve.

The open channel may extend along a direction of insertion of the analyte sensor, specifically the open channel is a straight channel. The direction of insertion may be transverse, specifically essentially perpendicular, to a skin site of the user. The direction of insertion may be transverse, specifically essentially perpendicular, relative to the lower side and to the upper side of the housing. Further, the open channel may extend transversely, specifically essentially perpendicularly, to a direction of extension of the housing. The open channel may extend transversely, specifically essentially perpendicularly, relative to the lower side and to the upper side of the housing. The direction of insertion may correspond to a direction of extension of the open channel. The open channel may specifically have an upper side opening and an opposing lower side opening. The upper side opening may be located on the upper side of the housing facing away from the skin site and the lower side opening may be located on the lower side of housing facing the skin site.

As outlined above, the open channel at least partially surrounds the analyte sensor and the insertion component. Specifically, the open channel may at least partially circumferentially surround the analyte sensor and the insertion component, specifically at least the insertion cannula of the insertion component and, optionally also a part of the insertion cannula holder of the insertion component. The open channel may form a compartment, specifically a compartment for at least partially receiving the analyte sensor and the insertion component. The housing may be at least partially formed as a cylindrical ring at least partially surrounding the analyte sensor and the insertion component, specifically at least the insertion cannula of the insertion component and/or a part of the insertion cannula holder of the insertion component.

Specifically, the insertion component and the analyte sensor may be at least partially received within the open channel. The insertable portion of the analyte sensor may extend downwardly inside the open channel beyond the lower side of the housing. Further, the insertable portion of the insertion cannula may extend downwardly inside the open channel beyond the lower side of the housing. The insertion cannula holder may optionally be at least partially surrounded by the open channel. Thus, the insertion cannula holder may, optionally, at least partially protrude into the open channel, specifically from the upper side of the connector unit. The insertion cannula holder may be at least partially received inside the open channel. Optionally, the insertion cannula holder may extend downwardly inside the open channel beyond the lower side of the housing, specifically in case the insertion cannula holder is connected to the removable sterility cap. Further details are given below.

The insertion cannula holder may be at least partially arranged on the upper side of the housing. The insertion cannula holder may be configured for sealing the upper side opening of the open channel. The removable sterility cap may be at least partially arranged on the lower side of the housing. The removable sterility cap may be configured for sealing the lower side opening of the open channel. The insertion cannula holder may seal with the upper side opening of the open channel and the removable sterility cap may seal with the lower side opening of the open channel.

As outlined above, the housing further comprises the electronics compartment with the electronics unit received therein.

The term “compartment” as used herein is a broad term and is to be given its ordinary and customary meaning to a person of ordinary skill in the art and is not to be limited to a special or customized meaning. The term specifically relates to an arbitrary subpart of a superior element creating a partially or fully enclosed space that may be usable to contain and/or store objects. The subpart may specifically be completely or at least to a large extent closed such that an interior of the compartment may be isolated from a surrounding environment. Exemplarily, the compartment may be separated from other parts of the superior element by one or more walls. Thus, within the continuous analyte monitoring device, two or more compartments may be comprised which may fully or partially be separated from one another by one or more walls of the continuous analyte monitoring device. Each compartment may comprise a continuous space or lumen configured for receiving one or more objects.

The term “electronics compartment” as used herein is a broad term and is to be given its ordinary and customary meaning to a person of ordinary skill in the art and is not to be limited to a special or customized meaning. The term specifically relates to an arbitrary compartment which is configured for receiving an element or a combination of elements which fulfill an electrical or electronic purpose. The electronics unit may be positioned within the electronics compartment of the housing, specifically fixedly positioned.

The term “electronics unit” (used synonymously herein with “electronics” and “electronics assembly”) as used herein is a broad term and is to be given its ordinary and customary meaning to a person of ordinary skill in the art and is not to be limited to a special or customized meaning. The term specifically relates to an arbitrary device which is configured for performing at least one electronic function. Specifically, the electronics unit may have at least one electronic component. Specifically, the electronics unit may comprise at least one electronic component for one or more of performing a measurement with the analyte sensor, performing a voltage measurement, performing a current measurement, recording sensor signals, storing measurement signals or measurement data, transmitting sensor signals, and measurement data to another device. The electronics unit may specifically be embodied as a transmitter or may comprise a transmitter, for transmitting data. Other embodiments of the electronic components are feasible. These electronic components generally are known in the art of long-term monitoring one or more analytes, such as in from one or more of the above-mentioned prior art documents.

The electronics unit may comprise at least one circuit carrier, preferably a printed circuit board, more preferably a flexible printed circuit board. The term “circuit carrier” as used herein is a broad term and is to be given its ordinary and customary meaning to a person of ordinary skill in the art and is not to be limited to a special or customized meaning. The term specifically relates to an element or a combination of elements which are capable of carrying one or more electronic components and of interconnecting these one or more electronics components, such as interconnecting the one or more electronics components electrically or electronically with each other and/or with one or more contact pads. As an example, the circuit carrier may comprise a base and one or more electrical traces and/or one or more electrical contact pads disposed thereon and/or therein. The base, as an example, may be a flat element with a lateral extension which exceeds its width by at least a factor of 10, more preferably by at least a factor of 100 or even a factor of 1000. Other embodiments are feasible. Rigid materials which may be used for the base may be fiber-enforced plastic materials such as fiber-enforced epoxy materials like glass-fiber-enforced epoxy materials such as FR-4. Other materials may be used. Specifically, the base may be a flexible base, such that the circuit carrier may fully or partially be embodied as a flexible printed circuit board. In this case, as an example, the flexible base may fully or partially be made of one or more flexible plastic materials such as one or more plastic foils or laminate, such as polyimides.

An electronic component may be attached to the circuit carrier. The term “electronic component” may generally refer to an arbitrary element or combination of elements which fulfill an electrical or electronic purpose. Specifically, the electronic component may be or may comprise at least one component selected from the group consisting of an integrated circuit, an amplifier, a resistor, a transistor, a capacitor, a diode or an arbitrary combination thereof. The electronic component specifically may be or may comprise a device capable of controlling the analyte sensor, in order to perform an analytical measurement with the analyte sensor. Specifically, the device may comprise a voltage measurement device and/or a current measurement device. Other setups or embodiments are feasible. The electronic component, as an example, may comprise an application-specific integrated circuit (ASIC).

Therein, the electronic component may directly or indirectly be attached to the circuit carrier. The circuit carrier may be a printed circuit board, particularly a flexible printed circuit board. As an example, the electronic component may directly be attached to the circuit carrier by using one or more of soldering, bonding or electrically conductive adhesive. Thus, the circuit carrier may comprise one or more contact pads, wherein corresponding contacts of the electronic component are electrically connected to the one or more contact pads. Additionally or alternatively, however, the electronic component may indirectly be attached to the circuit carrier, such as via an electronic housing. Thus, the electronic housing may be attached to the circuit carrier. Still, an electrical contact between the electronic component and the circuit carrier may be made, such as via a contact passing through the electronic housing. The electronic housing may fully or partially surround the electronic component. As an example, the electronic housing may comprise a lower electronic housing component attached to the circuit carrier, wherein the electronic devices inserted into the lower electronic housing component on a side opposing the circuit carrier. The electronic housing may further comprise a further electronic housing component, such as an upper electronic housing component, which, in conjunction with the lower electronic housing component, may form an encapsulation which fully or partially surrounds the electronic component. Additionally or alternatively, however, other types of encapsulation of the electronic component may be used, such as encapsulation by using one or more casting and/or potting compounds. Thus, as an example, the lower electronic housing component may be used for receiving the electronic component, wherein the upper shell or protection above the electronic component is created by using a casting and/or potting, such as by using one or more of an epoxy, a thermoplastic polymer, a rather, a silicone, and epoxies or the like. Additionally or alternatively, no electronic housing component may be used at all, such as by directly placing the electronic component onto the circuit carrier.

Specifically, the electronics unit may comprise an electrical energy reservoir, specifically a battery. The term “battery” as used herein is a broad term and is to be given its ordinary and customary meaning to a person of ordinary skill in the art and is not to be limited to a special or customized meaning. The term specifically relates to an arbitrary source of electric power comprising one or more electrochemical cells with external connections for powering an electrical device. When a battery supplies power, its positive terminal may be referred to as cathode and its negative terminal may be referred to as anode. The battery may specifically be a primary battery. The primary battery may be configured for being used once. The primary battery may also be referred to as single-use or disposable battery.

As outlined above, the housing is at least partially formed by the lower housing portion and the upper housing portion. The terms “lower housing portion” and “upper housing portion” may be considered as description without specifying an order and without excluding a possibility that several kinds of lower housing portions and upper housing portions may be applied. The upper housing portion and the lower housing portion may form an encapsulation for electronic components of the electronics unit. The upper housing portion and the lower housing portion may be connected to each other via at least one connection. Further details are given below in more detail.

Specifically, the upper housing portion may refer to a distal housing portion of the housing. The term “distal housing portion” as used herein is a broad term and is to be given its ordinary and customary meaning to a person of ordinary skill in the art and is not to be limited to a special or customized meaning. The term specifically may refer, without limitation, to an indication of a position of the portion of the housing in relation to a user which is furthermost away from a skin site of the user and/or which faces away from the skin. Exemplarily, for inserting the analyte sensor, the housing may be brought into contact with the skin site of the user. The distal housing portion may refer to a housing portion being distanced to the skin site of the user.

Specifically, the lower housing portion may refer to a proximal housing portion of the housing. The term “proximal housing portion” as used herein is a broad term and is to be given its ordinary and customary meaning to a person of ordinary skill in the art and is not to be limited to a special or customized meaning. The term specifically may refer, without limitation, to an indication of a position of the housing portion of the housing in relation to a user which is closest to a skin site of the user and/or which faces the skin. Exemplarily, for inserting the analyte sensor, the housing may be brought into contact with the skin site of the user. The proximal housing portion may refer to a housing portion being in close proximity to or even in direct contact with to the skin site of the user.

The housing may further comprise a connector unit comprising the open channel of the housing. The term “connector unit” (also referred to herein as simply “connector”) as used herein is a broad term and is to be given its ordinary and customary meaning to a person of ordinary skill in the art and is not to be limited to a special or customized meaning. The term specifically relates to an arbitrary device which is configured for connecting one object with another object. Specifically, the connector unit may be configured for connecting the sterile compartment with the electronics compartment. Specifically, the connector unit may have a channel and the analyte sensor may pass through the channel. The channel may also be referred to as opening. The analyte sensor may be partially received in the sterile compartment, specifically inside the open channel, and partially received in the electronics compartment. More specifically, the analyte sensor may be partially received in the sterile compartment, specifically inside the open channel, partially received within the channel of the connector unit and partially received in the electronics compartment. The channel may specifically be a sealed channel. The term “sealed” may generally refer to a property of an arbitrary element of being completely or at least to a large extent isolated from a surrounding environment. Specifically, the channel may be sealed with the sealing material. Further details are given below.

The open channel may be sealed off from the electronics compartment by a wall of the open channel. The wall of the open channel may be formed by the connector unit. Specifically, the connector unit may have a connector unit wall, specifically extending in the direction of insertion of the analyte sensor or extending transversely, specifically perpendicularly, to longitudinal sides of the housing. The connector unit wall may have a first side and an opposing second side. The first side may form a wall of the open channel. The first side may face an interior space of the open channel. Further, second side may face an interior space of the electronics compartment. The connector unit wall may be manufactured as one single piece or may be made of two or more components which may be arranged next to each other and/or may overlap with each other. The two or more components may be assembled and may optionally be bonded together such as with the sealing material and/or with another bonding material such as glue or the like.

The connector unit may have an upper connector unit side and a lower connector unit side. The open channel may connect the upper connector unit side and a lower connector unit side. The terms “upper connector unit side” and “lower connector unit side” may be considered as description without specifying an order and without excluding a possibility that several kinds of upper connector unit sides and lower connector unit sides may be applied. The upper connector unit side and the lower connector unit side of the connector unit may respectively have essentially flat surfaces.

Specifically, the upper connector unit side may refer to a distal connector unit side of the connector unit. The term “distal connector unit side” as used herein is a broad term and is to be given its ordinary and customary meaning to a person of ordinary skill in the art and is not to be limited to a special or customized meaning. The term specifically may refer, without limitation, to an indication of a position of the side of the connector unit in relation to the user which is furthermost away from the skin site of the user. Exemplarily, for inserting the analyte sensor, the connector unit may be brought into contact with the skin site of the user. The distal connector unit side may refer to a side being distanced to the skin site of the user.

Specifically, the lower connector unit side may refer to a proximal side of the connector unit. The term “proximal connector unit side” as used herein is a broad term and is to be given its ordinary and customary meaning to a person of ordinary skill in the art and is not to be limited to a special or customized meaning. The term specifically may refer, without limitation, to an indication of a position of the side of the connector unit in relation to a user which is closest to a skin site of the user. Exemplarily, for inserting the analyte sensor, the connector unit may be brought into contact with the skin site of the user. The proximal connector unit side may refer to a side being in close proximity to or even in direct contact with to the skin site of the user.

The open channel and the channel may be arranged transversely, specifically essentially perpendicularly, to each other. The channel may be formed by a hole or by a cutout within the connector unit, specifically in a wall of the connector unit. The channel itself may be embodied as an essentially straight channel. With regard to a definition of the terms “channel” and “straight,” reference to the description above is made.

The analyte sensor may comprise an in vivo proximal portion and an ex vivo distal portion. The in vivo proximal portion may correspond to the insertable portion of the analyte sensor as described above. The in vivo proximal portion may be configured for being inserted into the body tissue of the user. The ex vivo distal portion may be configured for staying outside of the body tissue of the user. The ex vivo distal portion and the in vivo proximal portion may be arranged transversely, specifically essentially perpendicularly, to each other. The in vivo proximal portion may extend along the direction of insertion. The ex vivo distal portion may comprise a first section being received in the channel and a second section being received in the electronics compartment. The electronics unit may be electrically connected to the analyte sensor. Thus, specifically, the second section of the ex vivo distal portion may be electrically connected to an electronics component of the electronics unit.

Specifically, the first section and the second section of the ex vivo distal portion may be essentially arranged along a straight line. Alternatively, the first section and the second section ex vivo distal portion may be arranged in an angle of 5° to 90°, specifically of 20° to 80°, more specifically of 40° to 70°, to each other. Specifically, the first section and the second section of the ex vivo distal portion may comprise a bend in an angle of 5° to 90°, specifically of 20° to 80°, more specifically of 40° to 70°, to each other. Specifically, the bend may be arranged in a plane parallel to the side of the base plate attachable to the skin. Thereby, a positioning and stabilization of the analyte sensor within the connector unit and/or within the electronics compartment may be improved.

The connector unit may specifically form an intermediate component. The term “intermediate component” as used herein is a broad term and is to be given its ordinary and customary meaning to a person of ordinary skill in the art and is not to be limited to a special or customized meaning. The term specifically may refer, without limitation, to an arbitrary component or compartment between at least two other compartments and/or which may be located in another compartment. Thus, the intermediate component may form part of the sensor compartment and may be sealed from the electronics compartment. The intermediate component may be or may comprise an intermediate compartment and/or, as an example, a sealing ring or a ring-shaped element. Specifically, the electronics compartment may at least partially surround the intermediate component. The intermediate component may be at least partially designed as a cylindrical ring at least partially surrounding the analyte sensor and/or the insertion component. The removable sterility cap and the insertion cannula holder may be separated by the intermediate component and may both be removably connected to the intermediate component.

The connector unit may specifically comprise a lower part and an upper part. The lower part and the upper part, in conjunction may form the connector unit. Exemplarily, one or both of the lower part and the upper part may be or may comprise a sealing ring or a ring-shaped element. The terms “upper part” and “lower part” may be considered as description without specifying an order and without excluding a possibility that several kinds of upper parts and lower parts may be applied.

Specifically, the upper part may refer to a distal part of the connector unit. The term “distal part” as used herein is a broad term and is to be given its ordinary and customary meaning to a person of ordinary skill in the art and is not to be limited to a special or customized meaning. The term specifically may refer, without limitation, to an indication of a position of the part of the connector unit in relation to the user which is furthermost away from the skin site of the user. Exemplarily, for inserting the analyte sensor, the connector unit may be brought into contact with the skin site of the user. The distal part may refer to a part being distanced to the skin site of the user or facing away from the skin.

Specifically, the lower part may refer to a proximal part of the connector unit. The term “proximal part” as used herein is a broad term and is to be given its ordinary and customary meaning to a person of ordinary skill in the art and is not to be limited to a special or customized meaning. The term specifically may refer, without limitation, to an indication of a position of the part in relation to a user which is closest to a skin site of the user. Exemplarily, for inserting the analyte sensor, the part may be brought into contact with the skin site of the user. The proximal part may refer to a part being in close proximity to or even in direct contact with to the skin site of the user, or facing the skin.

An upper section of the open channel may be formed by the upper part of the connector unit and a lower section of the open channel may be formed by the lower part of the connector unit. Further, the wall of the open channel may be at least partially formed by the lower part of the connector unit and by the upper part of the connector unit.

The housing may comprise a base plate being configured for attachment to a skin site of a user, specifically via at least one adhesive. Further, the housing may comprise an upper plate. The base plate and the upper plate are configured for forming at least parts of the longitudinal sides of the electronics compartment. Specifically, the base plate may comprise a lower surface configured for being placed on a user's skin. The lower surface may exemplarily have a shape of a circular ring surrounding the analyte sensor. More specifically, the base plate may comprise an adhesive surface for attachment to the user's skin. The term “adhesive surface” as used herein is a broad term and is to be given its ordinary and customary meaning to a person of ordinary skill in the art and is not to be limited to a special or customized meaning. The term specifically relates to a surface being capable to bind to an object and to resist separation. Exemplarily, the adhesive surface may comprise a plaster or an adhesive strip. The plaster or the adhesive strip may comprise an adhesive material. The adhesive surface may be directly or indirectly attached to the housing.

The lower part of the connector unit and the base plate may specifically form a single piece. The lower part of the connector unit and the base plate may be permanently built into each other or may be manufactured in one single piece. Thus, specifically, the lower part of the connector unit may be formed by a wall, specifically by an at least partially circumferential wall protruding from the base plate.

The upper part of the connector unit and the upper plate may specifically form a single piece. The upper part of the connector unit and the upper plate may be permanently built into each other or may be manufactured in one single piece. Thus, specifically, the upper part of the connector unit may be formed by a wall, specifically by an at least partially circumferential wall protruding from the upper plate.

However, also other embodiments may be possible. Thus, specifically, the lower part of the connector unit and the base plate may form a single piece and the upper plate and the upper part of the connector unit may be provided as separate pieces. Alternatively, the upper part of the connector unit and the upper plate may form a single piece and the base plate and the lower part of the connector unit may be provided as separate pieces. Alternatively, the upper part of the connector unit, the lower part of the connector unit, the upper plate and the base plate may respectively be provided as separate pieces.

As outlined above, the base plate and the upper plate may be configured for forming at least parts of the longitudinal sides of the electronics compartment. Specifically, the base plate and the upper plate, in conjunction may form an encapsulation for the electronics unit. Specifically, the electronics compartment may be formed by the base plate, the upper plate and the connector unit. The longitudinal sides of the electronics compartment may be formed by the base plate and the upper plate and, optionally, also by longitudinal sides of the connector unit, specifically of the upper part and/or of the lower part of the connector unit. The lower side of the housing may be formed by the base plate and, optionally also by the lower connector unit side of the connector unit. The adhesive surface may specifically be a surface of the base plate and, optionally, also a surface of the connector unit. The upper side of the housing may be formed by the upper plate and, optionally also by the upper connector unit side of the connector unit.

Specifically, the lower connector unit side of the connector unit may be essentially flush with a lower side of the base plate. Thus, the lower side of the base plate and the lower connector unit side of the connector unit may form an essentially flat surface. Optionally, the upper connector unit side of the connector unit may be essentially flush with an upper side of the upper plate. Thus, the upper connector unit side of the connector unit and the upper side of the upper plate may form an essentially flat surface.

Further, shorter sides of the electronics compartment facing the outer environment of the continuous analyte monitoring device may be formed at least partially by the base plate and/or by the upper plate.

As outlined above, the removable sterility cap, the open channel and the insertion cannula holder form the sterile compartment for the insertion cannula and at least the insertable portion of the analyte sensor. Thus, the insertion cannula and at least the insertable portion of the analyte sensor may be received in the sterile compartment. The sterile compartment may also be referred to as sensor compartment.

The sterile compartment may be a sealed compartment. The term “sealed compartment” as used herein is a broad term and is to be given its ordinary and customary meaning to a person of ordinary skill in the art and is not to be limited to a special or customized meaning. The term specifically may refer, without limitation, to a compartment being isolated from a surrounding environment such that a transfer of gas, fluids and/or solid elements is completely or at least to a large extent reduced. The term “sterile compartment” as used herein is a broad term and is to be given its ordinary and customary meaning to a person of ordinary skill in the art and is not to be limited to a special or customized meaning. The term specifically may refer, without limitation, to an arbitrary compartment configured to provide a sterile packaging for object received within the sterile compartment. The term “sterile” as used herein is a broad term and is to be given its ordinary and customary meaning to a person of ordinary skill in the art and is not to be limited to a special or customized meaning. The term specifically may refer, without limitation, to a property of an arbitrary object of being at least to a large extent free from all forms of life and/or other biological agents such as prions, viruses, fungi, bacteria or spore forms and also of ingress of liquid, moisture, and dust. Thus, the sterile object may be treated by a sterilization process that eliminates and/or deactivates the forms of life and/or the other biological agents. The sterilization process may comprise one or more of the following techniques: heating, chemical treatment including treatment by gas, irradiation, high pressure, and filtration. However, other techniques are feasible. The sterilization process may be conducted within a specified region or area of the object such as a surface of the object. Specifically, the sterilization process may be carried out with gas sterilization using generally known gas such as ethylene oxide (EtO) gas or vaporized hydrogen peroxide.

Embodiments of a medical analyte sensor device comprising an analyte sensor, a cannula, a cannula holder, a sterility cap in a sterility compartment and an insertion aid are disclosed in EP 3 202 324 A1, EP 3 727 130 A1, EP 3 988 014 A1 and EP 3 202 323 A1, which are herewith incorporated by reference.

The removable sterility cap and/or the insertion cannula holder may be reversibly or irreversibly connected to the housing, specifically to the connector unit, and/or to each other.

As outlined above, the housing may comprise the upper side and the lower side and the open channel may connect the upper side and the lower side. The removable sterility cap may be at least partially located on the lower side of the housing, specifically of the connector unit, and at least a part of the insertion cannula holder may extend from the upper side of the housing, specifically of the connector unit. The insertion cannula holder and the removable sterility cap may be respectively removably connected to the housing, specifically to the connector unit, specifically on opposing sides of the housing, specifically of the connector unit, more specifically of the open channel. The insertion cannula holder may seal with the upper side opening of the open channel and the removable sterility cap may seal with the lower side opening of the open channel.

Specifically, the removable sterility cap may at least partially be arranged on the lower side of the housing, specifically of the connector unit. The removable sterility cap may be removably connected, specifically attached, to the lower side of the housing, specifically of the connector unit. Further, additionally or alternatively, the removable sterility cap and the insertion cannula holder may be reversibly or irreversibly connected to each other. The removable sterility cap may be configured for removal before insertion of the insertable portion of the analyte sensor into the body tissue.

The insertion cannula holder may be at least partially arranged on the upper side of the housing, specifically of the connector unit, and may optionally additionally be at least partially located within the open channel. The insertion cannula holder may be removably connected, specifically attached, to the upper side of the housing, specifically of the connector unit. Further, the insertion cannula holder may be configured for closing and/or sealing the upper side opening of the open channel. Optionally, the insertion cannula holder may pass through the lower side opening of the open channel, specifically in order for establishing a connection to the removable sterility cap. Specifically, the insertion cannula holder may be at least partially received inside the open channel. The insertion cannula holder may be configured for removal after insertion of the insertable portion of the analyte sensor into the body tissue.

Specifically, the insertion cannula holder may form a removable cap, specifically a removable upper cap. The insertion cannula holder may be configured for sealing an end of the open channel of the housing, specifically of the connector unit, specifically of the upper side opening of the open channel.

Thus, the removable sterility cap may also be referred to as removable lower cap and the insertion cannula holder may also be referred to as removable upper cap. The terms “upper cap” and “lower cap” may be considered as description without specifying an order and without excluding a possibility that several kinds of upper caps and lower caps may be applied. The removable lower cap and the removable upper cap may be at least partially arranged or at least partially located on opposing sides of the connector unit, specifically of the open channel.

The insertion cannula holder may be removably connected to the housing, specifically to the connector unit, specifically to a surface of the housing, specifically of the connector unit, and/or to the removable sterility cap via a connection, specifically via at least one of a screwing connection, a bayonet connection. The removable sterility cap may be removably connected to the housing, specifically to the connector unit, specifically to the surface of the housing, specifically of the connector unit, and/or to the insertion cannula holder via a connection, specifically via at least one of a force fit connection, a form fit connection, a screwing connection, a magnetic connection or a bayonet connection. Specifically, the insertion cannula holder may be reversibly or irreversibly connected to a surface of the upper side of the housing, specifically of the connector unit, and/or to the removable sterility cap. Specifically, the removable sterility cap may be reversibly or irreversible connected to a surface of the lower side of the housing, specifically of the connector unit, and/or to insertion cannula holder.

Specifically, the removable sterility cap may be configured to be pulled off from the housing, specifically of the connector unit and/or from the insertion cannula holder. Further, specifically, the insertion cannula holder may be configured to be pulled off from the housing, specifically from the connector unit and/or from the removable sterility cap. Thus, the removable sterility cap and/or the insertion cannula holder may, in a stage connected to the housing, specifically to the connector unit, overlap with the housing, specifically to the connector unit, or vice a versa. Additionally or alternatively, the removable sterility cap may overlap with the insertion cannula holder or vice a versa. The housing, specifically the connector unit, specifically may comprise a guiding surface for guiding the removable sterility cap or the insertion cannula holder during pulling off the removable sterility cap or the insertion cannula holder. Additionally or alternatively, the removable sterility cap may comprise a guiding surface for guiding the insertion cannula holder during pulling off the insertion cannula holder from the removable sterility cap or vice a versa. Thus, the insertion cannula holder may comprise a guiding surface for guiding removable sterility cap during pulling off the removable sterility cap from the insertion cannula holder.

Further, specifically, the insertion cannula holder may be removably connected to the housing, specifically to the connector unit, at an upper predetermined breaking point and/or the removable sterility cap may be removably connected to the housing, specifically to the connector unit, at a lower predetermined breaking point. As further used herein, the term “predetermined breaking point” may refer to an arbitrary part of an element being configured to break during mechanical load while other parts of the element remain undamaged. Specifically, the predetermined breaking point may comprise a notch wherein a thickness of the element may be smaller in comparison to other parts of the element. The upper predetermined breaking point and/or the lower predetermined breaking point may specifically be ring-shaped breaking points. The terms “upper breaking point” and “lower breaking point” may be considered as description without specifying an order and without excluding a possibility that several kinds of upper breaking points and lower breaking points may be applied.

At least one of the removable sterility cap and the insertion cannula holder may comprise a hygroscopic material, preferably a desiccant, more preferably activated carbon.

As outlined above, the housing comprises the sealing channel comprising the input filling port and the output filling port.

The term “sealing channel” as used herein is a broad term and is to be given its ordinary and customary meaning to a person of ordinary skill in the art and is not to be limited to a special or customized meaning. The term specifically relates to an arbitrary element which may have an elongate shape and which may provide a free volume or lumen configured for being filled with a sealing material.

The sealing channel may be partially formed by the lower housing portion and may be partially formed by the upper housing portion. Exemplarily, the sealing channel may be partially formed by a groove within the lower housing portion, specifically within a surface of the lower housing portion, and/or by a groove within the upper housing portion, specifically within a surface of the upper housing portion. The groove within the lower housing portion and/or the groove within the upper housing portion may specifically have a shape selected from the group consisting of: a semi-circular shape; a semi-oval shape; a polygonal shape, a trapezoidal shape, specifically a rectangular shape. However, also other shapes may be possible. Exemplarily, the sealing channel may be partially formed by the groove within the lower housing portion and by the groove within the upper housing portion. Further, exemplarily, the sealing channel may be partially formed by the groove within the lower housing portion and by a flat surface of the upper housing portion. Further, exemplarily, the sealing channel may be partially formed by the groove within the upper housing portion and by a flat surface of the lower housing portion. Also other embodiments may be feasible.

The groove within the lower housing portion and/or the groove within the upper housing portion may respectively form at least one open channel. The term “open channel” as used herein is a broad term and is to be given its ordinary and customary meaning to a person of ordinary skill in the art and is not to be limited to a special or customized meaning. The term specifically relates to a slot or a trench cut into a surface of a solid material, specifically into a smooth surface of the solid material. The upper housing portion may form a cover for the groove within the lower housing portion and vice versa. Thus, the lower housing portion may form a cover for the groove within the upper housing portion. The term “cover” as used herein is a broad term and is to be given its ordinary and customary meaning to a person of ordinary skill in the art and is not to be limited to a special or customized meaning. The term specifically relates to an arbitrary element which has a surface configured to be attachable to a further surface of a further element. Thus, a shape of the surface of the cover and a shape of the further surface of the further element may be at least partially be complementary to each other. Exemplarily, the surface of the cover and the further surface of the further element may at least partially be embodied as flat surfaces such that a tight connection between the surface and the further surface may be formable. The lower housing portion and the upper housing portion, specifically the groove within the lower housing portion and the groove within the upper housing portion, or the groove within the lower housing portion and the cover of the upper housing portion, or the groove within the upper housing portion and the cover of the lower housing portion, may have angled overlaps. Thus, a tolerance gap may be bridged when the lower housing portion and the upper housing portion are assembled, specifically pressed together.

The sealing channel may specifically be an at least partially circumferential sealing channel. Specifically, the sealing channel may specifically be a circumferential sealing channel. The term “circumferential channel” as used herein is a broad term and is to be given its ordinary and customary meaning to a person of ordinary skill in the art and is not to be limited to a special or customized meaning. The term specifically relates to an arbitrary channel which may surround an object or a part of an object in at least two dimensions. The sealing channel may specifically be embodied as a closed channel, e.g., as a channel without ends. The sealing material may be filled into the sealing channel via the inlet filling port. Further details are given below. The sealing channel may be at least partially shaped as a torus. The sealing channel, specifically in a top view of the sealing channel, may be formed as a ring. In a top view on the groove within the upper housing portion and/or on the grove within the lower housing portion, the grooves may have a shape of a ring, specifically of a circular ring. Exemplarily, the sealing channel, specifically a cross-sectional area of the sealing channel, more specifically a cross-sectional area of the sealing channel transverse to a direction of extension of the sealing channel, may have a mean diameter or a width of 0.1 mm to 5 mm, specifically of 0.2 mm to 2 mm, more specifically of 0.4 mm to 1.6 mm. However, also other dimensions may be possible.

At least one of: a width of a lumen of the sealing channel; a shape of the sealing channel, specifically a shape of a cross-section of the sealing channel; a dimension of the sealing channel, specifically a dimension of the cross-section of the sealing channel; may be constant along the sealing channel. Alternatively, at least one of: the width of the lumen of the sealing channel; the shape of the sealing channel, specifically the shape of the cross-section of the sealing channel; the dimension of the sealing channel, specifically the dimension of the cross-section of the sealing channel; may vary along the sealing channel. A change of the width of the lumen of the sealing channel may specifically refer to a change in a dimension of a cross-sectional area of the sealing channel transverse to the direction of extension of the sealing channel. Exemplarily, the cross-sectional area of the sealing channel transverse to the direction of extension of the sealing channel may have an elliptical basic shape and at least one of a dimension of the main axis and a dimension of a secondary axis may vary along the sealing channel.

At least one of: the width of the lumen of the sealing channel; the dimension of the sealing channel, specifically the dimension of the cross-section of the sealing channel; a cross-sectional area of the sealing channel, specifically a cross-sectional area of the sealing channel transverse to the direction of extension of the sealing channel; may be larger in an area close to the sealing channel intersecting with the channel than in an area being further away from the sealing channel intersecting with the channel. Specifically, at least one of: the width of the lumen of the sealing channel; the dimension of the sealing channel, specifically the dimension of the cross-section of the sealing channel; a cross-sectional area of the sealing channel, specifically the cross-sectional area of the sealing channel transverse to the direction of extension of the sealing channel; may decrease, specifically continuously, the further a section of the sealing channel is away from the channel.

Specifically, the sealing channel may be divided into a first arm and into a second arm by the input filling port and by the output filling port. The first arm may intersect with the channel. At least one of: the width of the lumen of the sealing channel; the dimension of the sealing channel, specifically the dimension of the cross-section of the sealing channel; a cross-sectional area of the sealing channel, specifically the cross-sectional area of the sealing channel transverse to the direction of extension of the sealing channel; may be larger in the first arm than in the second arm. More specifically, at least one of: a mean width of the lumen of the sealing channel; a mean dimension of the sealing channel, specifically a mean dimension of the cross-section of the sealing channel; a mean cross-sectional area of the sealing channel, specifically a mean cross-sectional area of the sealing channel transverse to the direction of extension of the sealing channel; may be larger in the first arm than in the second arm.

At the intersection of the channel with the sealing channel, the analyte sensor may impose an increased mechanical resistance to a flow of the sealing material, specifically as it fills up the sealing channel and/or the channel. To avoid that the sealing material only flows along an arm of the sealing channel in the area being further away from the sealing channel, the lumen of the sealing channel may be increased such that differences in flow resistance can be balanced out. This way, this disclosure may be associated with the surprising advantage of improved filling of the sealing channel and/or the channel with the sealing material. This in turn may improve sealing of the analyte sensor as it passes through the open channel from the sterile compartment into the electronics compartment.

As outlined above, the sealing channel comprises the input filling port and the output filling port. The term “port” as used herein is a broad term and is to be given its ordinary and customary meaning to a person of ordinary skill in the art and is not to be limited to a special or customized meaning. The term specifically relates to an arbitrary unit which may be configured to connect two or more elements to each other such that a fluid medium or a solid medium may be transferable from one element to another element. Specifically, a port may be a part of a channel or may form a channel comprising at least one opening which allows for fluid or solid medium entering the element or leaving the element and/or which allows for establishing a fluid connection between the two elements. The port may specifically be a tight port configured to prevent a leakage of the solid or fluid medium at least to a large extent.

The input filling port may be configured for filling the sealing channel with the sealing material. The output filling port may be configured for enabling a leakage of the sealing material, specifically of an excess of the sealing material from the sealing channel. The leakage of the sealing material from the sealing channel via the output filling port may be a desired leakage. Optionally, the housing may further comprise a cavity arranged between the sealing channel and the output filling port. Specifically, the cavity may be fluidically arranged between the sealing channel and the output filling port. The cavity may provide a free volume. The cavity may be configured for collecting the sealing material, specifically the excess of the sealing material from the sealing channel. Specifically through the usage of the cavity and, optionally also through a choice of a shape of the sealing channel, flow front velocities may be synchronized for different distances such that air may exit completely or at least to a large extent from the sealing channel, and, optionally, also from the channel. Specifically through the cavity and, optionally also through a choice of a shape of the sealing channel, flow front velocities may be synchronized such that the sealing material in all sealing channels converges into the cavity simultaneously, preventing an intrusion of one flow front into another sealing channel, eliminating a possibility of trapping air within the channels. A flow front may push the air in front of it, specifically preventing bubbles from forming in the sealing channel and/or in the channel.

The input filing port and/or the output filling port may be formed as a channel being fluidically connected to the sealing channel. The input filing port and/or the output filling port may be arranged transversely, specifically perpendicularly, to the sealing channel, specifically in a top view on the sealing channel. The input filing port and the sealing channel and/or the output filling port and the sealing channel may extend along one plane along a direction of extension of the housing. Alternatively, the input filing port and/or the output filling port may extend transversely, specifically perpendicularly to a plane along which the sealing channel extends. The input filling port and/or the output filling port may be accessible from the outer environment of the continuous analyte monitoring device or from an outer environment of the connector unit.

The input filling port and the output filling port may be arranged in a distance to each other. The input filling port may be distinct from the output filling port. Specifically, the input filling port and the output filling port may be arranged opposite to each other, specifically in the top view of the sealing channel. Specifically, the sealing channel may be divided by the input filling port and the output filling port in a first half and in a second half. The first half and the second half may be essentially equivalent.

Specifically, the sealing channel may be configured to be filled with the sealing material in an assembled state of the lower housing portion and the upper housing portion. In the assembled state of the lower housing portion and the upper housing portion, the sealing channel may be formed by the groove within the lower housing portion and/or by the groove within the upper housing portion. Further details in this regard are given above. In the assembled state of the lower housing portion and the upper housing portion, the sealing material may be filled into the sealing channel via the input filling port. An attachment of the lower housing portion and the upper housing portion to each other may be realized through filling the sealing material into the sealing channel. To the contrary, in current continuous glucose monitoring devices, a connection between housing parts is usually made using adhesive filled within grooves. Air bubbles can form in the adhesive when the adhesive is applied and/or air bubbles can form when the upper housing portion is not placed exactly perpendicularly on the lower housing portion. A housing part may be fitted from above and air may be displaced and has to escape through a ventilation hole.

The filling of the sealing channel with the sealing material may be accomplished by generally known methods and tools, e.g., by using an electrical pump coupled to a tubing or a syringe, which injects the sealing material into the input filling port of the sealing channel. In another embodiment, the sealing material is injected into the input filling port of the sealing channel while an under-pressure is applied to the output filling port, e.g., by a pump with a tubing or a syringe, wherein the under-pressure facilitates a flow of the sealing material throughout the sealing channel.

The housing may be at least partially formed as a ring, specifically as a cylindrical ring. The electronics compartment may be sealed from an outer environment by an encapsulation of the upper housing portion and the lower housing portion. The upper housing portion and the lower housing portion may be connected to each other at at least two different places. Specifically, the electronics compartment may be sealed from the outer environment at the at least two different places. Specifically, a first place may correspond to a sealing of the electronics compartment from the sterile compartment, specifically from the open channel. Further, specifically, a second place may corresponded to a sealing of the electronics compartment from the outer environment of the continuous analyte monitoring device.

Specifically, the sealing channel may be a circumferential sealing channel and the sealing channel may seal the electronics compartment from the sterile compartment. The sealing channel may surround, specifically circumferentially surround the open channel of the housing. The sealing channel may be located in a wall separating the electronics compartment from the sterile compartment. The sealing channel may be arranged between the sterile compartment, specifically the open channel, and the electronics compartment.

Further, the sealing channel may be a circumferential sealing channel and the sealing channel may seal the electronics compartment from the outer environment of the continuous analyte monitoring device. The sealing channel may surround, specifically circumferentially surround the electronics unit received in the housing. The sealing channel may be located in a wall separating the electronics compartment from the outer environment of the continuous analyte monitoring device.

Specifically, the housing may comprise two of the sealing channels. The two sealing channels may comprise an inner circumferential sealing channel and an outer circumferential sealing channel. The inner circumferential sealing channel may have a smaller diameter than the outer circumferential sealing channel.

The inner circumferential sealing channel may seal the electronics compartment from the sterile compartment. The inner circumferential sealing channel may surround, specifically circumferentially surround the open channel of the housing. The inner circumferential sealing channel may be located in a wall separating the electronics compartment from the sterile compartment. The inner circumferential sealing channel may be arranged between the sterile compartment, specifically the open channel, and the electronics compartment.

The outer circumferential sealing channel may seal the electronics compartment from the outer environment of the continuous analyte monitoring device. The outer circumferential sealing channel may surround, specifically circumferentially surround the electronics unit received in the housing. The outer circumferential sealing channel may be located in a wall separating the electronics compartment from the outer environment of the continuous analyte monitoring device.

Specifically, the outer circumferential sealing channel and the inner circumferential sealing channel may be embodied independently from each other. Specifically, each of the inner circumferential sealing channel and an outer circumferential sealing channel may respectively comprises one input filling port and one output filling port.

Further, specifically, the inner circumferential sealing channel and the outer circumferential sealing channel and the inner circumferential sealing channel may be connected to each other, specifically via a connecting channel. The connecting channel specifically be may configured for fluidically connecting the inner circumferential sealing channel and the outer circumferential sealing channel. The sealing material may be transferable from one of the inner circumferential sealing channel and the outer circumferential sealing channel to the other one of the inner circumferential sealing channel and the outer circumferential sealing channel and vice a versa via the connecting channel. The connecting channel may be embodied as an essential straight channel. The connecting channel may specifically be formed within the lower housing portion, specifically within the base plate. Specifically, the connecting channel may be formed as a groove within the lower housing portion. The groove may be located on a side of the lower housing portion facing the electronics compartment. The groove may be covered with a cover, specifically such that the connecting channel is formed. Specifically in case of the connecting channel, the housing may one input filling port and two output filling ports. Specifically, the input filling port may be arranged at the outer circumferential sealing channel. The input filling port may be fluidically connected to the outer circumferential sealing channel. Further, specifically, one of the two output filling ports may be arranged at the outer circumferential sealing channel and another one of the two output filling ports may be arranged at the inner circumferential sealing channel. Specifically, one of the two output filling ports may be fluidically connected to the outer circumferential sealing channel and another one of the two output filling ports may be fluidically connected to the inner circumferential sealing channel. As outlined above, in current continuous glucose monitoring devices, a connection between housing parts is usually made using adhesive filled within grooves. Air bubbles can form in the adhesive when the adhesive is applied and/or air bubbles can form when the upper housing portion is not placed exactly perpendicularly on the lower housing portion. In current continuous glucose monitoring devices, the adhesive is usually applied in the inner circumferential sealing channel and the outer circumferential sealing channel. By establishing a connection between the one input filling port and the two output filling ports, a whole injection process can be done through the input filling port. Thus, the current adhesive process can be reduced by one additional step of applying the adhesive in the inner circumferential sealing channel. The established connection may reduce process costs and process time.

As outlined above, the housing may comprise the connector unit comprising the open channel of the housing. The connector unit may be formed by the lower part of the connector unit and by the upper part of the connector unit. Specifically, the lower housing portion may be the lower part of the connector unit and the upper housing portion may by the upper part of the connector unit. Specifically, the sealing channel may be a circumferential sealing channel and the sealing channel may seal the electronics compartment from the sterile compartment. The sealing channel may be partially formed by the lower part of the connector unit and may be partially formed by the upper part of the connector unit. Exemplarily, the sealing channel may be partially formed by a groove within the lower part, specifically within a surface of the lower part, and/or by a groove within the upper part, specifically within a surface of the upper part. The sealing channel may be located between the lower part of the connector unit and the upper part of the connector unit. The sealing channel may surround, specifically circumferentially surround the open channel of the housing. The sealing channel may be located in a wall separating the electronics compartment from the sterile compartment. The sealing channel may be arranged between the sterile compartment, specifically the open channel, and the electronics compartment.

Further, as outlined above, the connector unit may have the channel and the analyte sensor may pass through the channel. Specifically, the analyte sensor may be partially received in the sterile compartment, partially received within the channel of the connector unit and partially received in the electronics compartment. The channel may have a first end and an opposing second end. The channel may connect the sterile compartment with the electronics compartment via the first end and the second end. The channel may be formed as a groove within the lower part and/or within the upper part. The sealing channel and the channel may extend along a same plane. Specifically in a top view of the sealing channel, the channel may be arranged transversely to the sealing channel.

Specifically, the channel may intersect the sealing channel. The channel may be fluidically connected to the sealing channel through the intersection of the sealing channel via the channel. Specifically, the channel may intersect the sealing channel between the first end of the channel and the second end of the channel. The sealing channel may be arranged between the first end of the channel and the second end of the channel. Specifically, the channel may be configured to be filled with the sealing material in an assembled state of the lower part and the upper part. Thus, by filling the sealing channel via the input filling port, the channel may be sealed with the sealing material as well. Specifically, in an assembled state and manufactured of the continuous analyte monitoring device, the channel is at least partially filled, preferably fully filled with the sealing material.

Further, specifically, the lower housing portion may be the base plate and the upper housing potion may be the upper plate. The sealing channel may seal the electronics compartment from the sterile compartment or the sealing channel may seal the electronics compartment from the outer environment of the continuous analyte monitoring device.

As outlined above, the sealing material is configured for sealing the interior space enclosed by the upper housing portion and the lower housing portion. Further, the sealing material may be configured for fixedly attaching the lower housing portion and the upper housing portion to each other. Thus, on the one hand, the sealing material may be configured for attaching the upper housing portion to the lower housing portion and vice versa by material engagement. The sealing material may specifically be a glue attaching the upper housing portion to the lower housing portion and vice versa. Further, on the other hand, sealing material may form an encapsulation or contribute to an encapsulation of the interior space enclosed by the upper housing portion and the lower housing portion. Thus, due to the sealing material, the interior space enclosed by the upper housing portion and the lower housing portion may be least to a large extent isolated from a surrounding environment, specifically such that a transfer of gas, fluids and/or solid elements is completely or at least to a large extent reduced.

The sealing material may be a liquid material which increases in viscosity or hardens after the sealing material has been introduced into the channel of choice. The sealing material may specifically be selected from the group consisting of: silicone, a glue, and adhesive. The adhesive may specifically be selected from the group consisting of: an acrylic adhesive, an epoxy adhesive, a polyurethane adhesive, a cyanoacrylate adhesive, a solvent-based adhesive. The acrylic adhesive may specifically be an UV glue such as Vitralit®. However, also other kinds of sealing material may be feasible.

In a further aspect of this disclosure, a continuous analyte monitoring system is disclosed. The continuous analyte monitoring system comprises the continuous analyte monitoring device as described above or as will further be described below in more detail.

Further, the continuous analyte monitoring system comprises an insertion device at least partially covering the continuous analyte monitoring device. The insertion device is configured for enabling a user to drive the insertion cannula into the body tissue and to insert the insertable portion of the analyte sensor.

The term “insertion device” as used herein is a broad term and is to be given its ordinary and customary meaning to a person of ordinary skill in the art and is not to be limited to a special or customized meaning. The term specifically relates to an arbitrary technical construction being configured to insert an object into another object. The insertion device may also be referred to as insertion aid. The insertion device may comprise an insertion mechanism. As further used herein, the term “mechanism” may refer to an arbitrary mechanism designed to transform input forces and movement into a desired set of output forces and movement. Specifically, the insertion mechanism may be configured such that the user may apply a force in a direction of insertion to the insertion cannula. Therefore, the insertion device may be configured to facilitate a handling of the continuous analyte monitoring device by the user and/or to reduce application errors. The insertion device may at least partially surround the continuous analyte monitoring device. Further, the insertion device may be at least partially coupled to the continuous analyte monitoring device, specifically to at least one of the insertion cannula holder and the removable sterility cap.

The insertion device may comprise a removable lower cover mechanically coupled to the removable sterility cap. As further used herein, the term “cover” may refer to an arbitrary element that completely or at least to a large extent closes an object. Specifically, the cover may be or may comprise a shell, particularly a half-shell, surrounding the housing and/or to the continuous analyte monitoring device. The removable lower cover may be configured such that a removal of the removable lower cover removes the removable sterility cap. The insertion device may further comprise a frame. The term “frame” may refer to an arbitrary element which may be configured to support other components of a physical construction. The frame may be displaceable on the skin of the user and may at least partially surround the housing, the analyte sensor, the insertion cannula, the removable sterility cap, the insertion cannula holder and/or the connector unit. The insertion device may further comprise an upper cover. The upper cover may be directly or indirectly coupled to one or both of the insertion cannula or the insertion cannula holder, such that a movement of the upper cover against the frame drives the insertion cannula. The terms “lower cover” and “upper cover” may be considered as description without specifying an order and without excluding a possibility that several kinds of lower covers and upper covers may be applied.

The removable lower cover may comprise a basis which is connected to a lower part of the removable sterility cap, exemplarily via a snap connection, an adhesive bonding and/or a longitudinal guide or transferring force. The basis may comprise gripping surfaces for removing the removable sterility cap. The basis may at the same time be a cover for the adhesive surface. This may lead to an extended shelf-life of the adhesive surface. By removing the lower cover the removable sterility cap may be opened, the insertion cannula and the analyte sensor may be exposed and the adhesive surface may be exposed at the same time.

The continuous analyte monitoring device may further comprise a retraction mechanism for retracting the insertion cannula after insertion of the insertable portion of the analyte sensor into the body tissue. The term “retraction mechanism” may generally refer to an arbitrary construction which is configured to move an object in an opposite direction of a direction in which the object may have been moved before the retraction mechanism is applied. Therefore, the retraction mechanism may comprise a retraction contact spring element. The retraction contact spring element may be biased in order to retract the insertion cannula from the body tissue. The retraction mechanism may at least partially be comprised within the insertion cannula holder and/or within the upper cover.

The continuous analyte monitoring system may further comprise:
a sensor controller which is coupled to the analyte sensor, wherein the sensor controller is configured to receive analyte sensor data from the analyte sensor; and
a remote control which is configured to receive sensor data from the sensor controller and to process and/or display sensor data.

The sensor controller may specifically comprise at least one data processing unit, such as a processor. Further, the sensor controller may comprise at least one volatile or non-volatile data storage. The sensor controller may comprise at least one interface configured for entering commands and/or for outputting information. The at least one interface may comprise a wired interface and/or a wireless interface for unidirectionally or bidirectionally exchanging data or commands, specifically between the sensor controller and at least one further device.

The sensor controller may be configured to communicate the analyte sensor data to the remote control. The term “communication” as used herein is a broad term and is to be given its ordinary and customary meaning to a person of ordinary skill in the art and is not to be limited to a special or customized meaning. The term specifically may refer, without limitation, to a process of transferring information. In particular, information from a computational device may be transferred such as by sending or outputting information, e.g., onto another device. Specifically, a communication interface may be provided. The communication interface may specifically provide means for transferring or exchanging information. In particular, the communication interface may provide a data transfer connection, e.g., Bluetooth, NFC, inductive coupling or the like.

Specifically, the continuous analyte monitoring device may comprise a user interface. The term “user interface” as used herein is a broad term and is to be given its ordinary and customary meaning to a person of ordinary skill in the art and is not to be limited to a special or customized meaning. The term may refer, without limitation, to an element or device which is configured for interacting with its environment, such as for the purpose of unidirectionally or bidirectionally exchanging information, such as for exchange of one or more of data or commands. For example, the user interface may be configured to share information with a user and to receive information by the user. The user interface may be a feature to interact visually with a user, such as a display, or a feature to interact acoustically with the user. The user interface, as an example, may comprise one or more of: a graphical user interface; a data interface, such as a wireless and/or a wire-bound data interface.

In a further aspect of this disclosure, a method of assembling the continuous analyte monitoring device according as described above or as will further be described below in more detail is disclosed.

The method comprises the method steps as given in the independent claims and as listed as follows. The method steps may be performed in the given order. However, other orders of the method steps are feasible. Further, one or more of the method steps may be performed in parallel and/or on a timely overlapping fashion. Further, one or more of the method steps may be performed repeatedly. Further, additional method steps may be present which are not listed.

The method comprises:
a) providing the lower housing portion;
b) mounting the analyte sensor onto the lower housing portion;
c) placing the upper housing portion onto the lower housing portion; and/or
d) injecting the sealing material into the sealing channel via the input filling port, specifically under pressure, such that the lower housing portion and the upper housing portion are fixedly attached to each other and such that the interior space enclosed by the upper housing portion and the lower housing portion is sealed.

As outlined above, the housing may comprise the connector unit comprising the lower part and the upper part. In case the lower housing portion may be the lower part of the connector unit and the upper housing portion may by the upper part of the connector unit the manufacturing of the continuous analyte monitoring device may exemplarily be conducted as follows: The lower part of the connector unit may be provided, optionally with the base plate of the housing. The analyte sensor may be received within the channel or within a part of the channel formed by the lower part of the connector unit. Thereafter, the upper part of the connector unit may be placed on top of the lower part of the connector unit. In the assembled state of the upper part of the connector unit and the lower part of the connector unit, the sealing channel and, optionally also the channel in case the channel intersects the sealing channel, may be filled with the sealing material. Further, the removable sterility cap and the insertion component may be mounted. Thus, a sterility unit may be formed. The sterility unit may comprise the connector unit, the removable sterility cap, the insertion component and the analyte sensor. The sterility unit may be sterilized. Thereafter, in case the lower part of the connector unit is provided with the base plate of the housing, the electronics unit may be mounted on the base plate and the upper plate may be mounted on the base plate. Optionally, the upper plate may be sealed with the base plate via a further sealing channel which is filled with the sealing material. Thereby, the further sealing channel may be distinct from the sealing channel. The further sealing channel may be fluidically disconnected from the sealing channel. The further sealing channel may have a separate input filling port and a separate output filling port. Alternatively, the upper plate and the base plate may be connected via one or more of a form-fit connection, a force-fit connection. Also other kinds of connection may be possible.

Alternatively, in case the lower housing portion may be the lower part of the connector unit and the upper housing portion may by the upper part of the connector unit the manufacturing of the continuous analyte monitoring device may exemplarily be conducted as follows: The lower part of the connector unit may be provided with the base plate of the housing. The analyte sensor may be received within the channel or within a part of the channel formed by the lower part of the connector unit. Thereafter, the upper part of the connector unit may be placed on top of the lower part of the connector unit. The electronics unit may be mounted on the base plate and connected to the analyte sensor. The upper plate of the housing may be placed on top of the base plate of the housing. The continuous analyte monitoring device may comprise the inner circumferential sealing channel and the outer circumferential sealing channel. In the assembled state of the upper part of the connector unit and the lower part of the connector unit and of the base plate of the housing and the upper plate of the housing, the inner circumferential sealing channel and the outer circumferential sealing channel may be filled with the sealing material. Further, the removable sterility cap and the insertion component may be mounted.

In case the lower housing portion may be the base plate and the upper housing portion may by the upper plate, the manufacturing of the continuous analyte monitoring device may exemplarily be conducted as follows: The lower part of the connector unit may be provided, optionally with the base plate of the housing. The analyte sensor may be received within the channel or within a part of the channel formed by the lower part of the connector unit. Thereafter, the upper part of the connector unit may be mounted on top of the lower part of the connector unit. The channel may be sealed. Further, the removable sterility cap and the insertion component may be mounted. Thus, a sterility unit may be formed. The sterility unit may comprise the connector unit, the removable sterility cap, the insertion component and the analyte sensor. The sterility unit may be sterilized. Thereafter, in case the lower part of the connector unit is provided with the base plate of the housing, the electronics unit may be mounted on the base plate and the upper plate may be placed on the base plate. In an assembled state of the base plate and the upper plate, the sealing channel may be filled with the sealing material.

The method may further comprise installing the continuous analyte monitoring device within a cavity of an insertion device, specifically such that a continuous analyte monitoring system is formed. For further details of the insertion device, reference is made to the description above. The continuous analyte monitoring system may correspond to the continuous analyte monitoring system as described above or as will further be described below in more detail.

During step d), an assembly of the lower housing portion and the upper housing portion may be tilted. Optionally, the housing may further comprise the cavity arranged between the sealing channel and the output filling port. For further details of the cavity, reference is made to the description above. Specifically, during step d), the assembly of the lower housing portion and the upper housing portion may be continuously tilted such that the cavity continuously represents a highest point at an end of a flow front of the sealing material through the sealing channel. Thus, air may escape from the sealing channel, specifically via the output filling port. By reducing the probability of air entering or staying within the sealing channel, a tightness of a bonded joint between the lower housing portion and the upper housing portion may be more reliable and production waste may be reduced.

In a further aspect of this disclosure, a method of using the continuous analyte monitoring device as described above or as will further be described below in more detail is disclosed.

The method comprises the method steps as given in the independent claims and as listed as follows. The method steps may be performed in the given order. However, other orders of the method steps are feasible. Further, one or more of the method steps may be performed in parallel and/or on a timely overlapping fashion. Further, one or more of the method steps may be performed repeatedly. Further, additional method steps may be present which are not listed.

The method comprises:
I. providing the continuous analyte monitoring device;
II. removing the removable sterility cap;
III. inserting the analyte sensor into a body tissue of the user; and
IV. removing the insertion cannula holder, thereby removing the insertion cannula from the continuous analyte monitoring device.

The proposed devices and methods provide many advantages over known devices and methods.

Instead of applying glue first and assembling upper housing portion and the lower housing portion afterwards, with this disclosure, it is possible to first assemble upper housing portion and the lower housing portion and to conduct the gluing afterwards. This is enabled by the filling ports and the respective channels. This results in a simplification of the assembling and to a more reliable and reproducible fixation of the parts. Furthermore, bubble free gluing may be achieved, specifically by the additional venting ports.

A sealed channel system may be created when the housing is assembled. Halves of the sealing channel and/or of the channel may be designed in such a way that they can bridge a tolerance gap when the housing is pressed together during assembly by means of angled overlaps. The sealed channel system may enable an injecting of the sealing material at only one position, specifically eliminating a need for a dispensing robot to move along the adhesive gap. Costs, specifically manufacturing costs, and process time may be reduced. At the same time, the channel system may be designed in such a way that a flow front pushes air in front of it, preventing bubbles from forming in the channel system. The flow front speeds may be synchronized for different distances so that the air may exit completely or at least to a large extent. This may specifically be made possible by a choice of a cross-section of the channel system and a use of the cavities, specifically of caverns, at a point where the flow fronts meet. By inclining the housing so that the cavities, specifically the caverns, always represent the highest point at the end of the flow front, the air may collect in the cavities and may escape from the output filling port. By reducing a probability of air entering the channel system, a tightness of a glued joint is ensured and production waste is reduced.

Summarizing and without excluding further possible embodiments, the following embodiments may be envisaged:
Embodiment 1: A continuous analyte monitoring device, comprising:
an analyte sensor comprising an insertable portion adapted for at least partially being inserted into a body tissue of a user, wherein the analyte sensor is configured for detecting an analyte in a body fluid of the user;
an insertion component comprising an insertion cannula and an insertion cannula holder, wherein the insertion cannula is attached to the insertion cannula holder, wherein the analyte sensor is at least partially placed inside the insertion cannula;
a removable sterility cap, wherein the removable sterility cap at least partially surrounds the insertable portion of the analyte sensor;
a housing, wherein the housing comprises an open channel which at least partially surrounds the analyte sensor and the insertion component, wherein the housing further comprises an electronics compartment with an electronic unit received therein; wherein the housing is at least partially formed by a lower housing portion and by an upper housing portion, wherein the housing comprises a sealing channel comprising an input filling port an output filling port, wherein the sealing channel is at least partially filled with a sealing material being configured for sealing an interior space enclosed by the upper housing portion and the lower housing portion;
   wherein the removable sterility cap, the open channel of the housing and the insertion cannula holder from a sterile compartment for the insertion cannula and at least the insertable portion of the analyte sensor.
Embodiment 2: The continuous analyte monitoring device according to the preceding embodiment, wherein the sealing channel is configured to be filled with the sealing material in an assembled state of the lower housing portion and the upper housing portion.
Embodiment 3: The continuous analyte monitoring device according to any one of the preceding embodiments, wherein the electronics unit is electrically connected to the analyte sensor.
Embodiment 4: The continuous analyte monitoring device according to any one of the preceding embodiments, wherein the analyte sensor is partially received in the sterile compartment, specifically inside the open channel, and partially received in the electronics compartment.
Embodiment 5: The continuous analyte monitoring device according to any one of the preceding embodiments, wherein the sealing channel is at least partially formed by a groove within the lower housing portion and/or by a groove within the upper housing portion.
Embodiment 6: The continuous analyte monitoring device according to the preceding embodiment, wherein groove within the lower housing portion and/or the groove within the upper housing portion has a shape selected from the group consisting of: a semi-circular shape; a semi-oval shape; a polygonal shape, a trapezoidal shape, specifically a rectangular shape.
Embodiment 7: The continuous analyte monitoring device according to any one of the preceding embodiments, wherein the sealing channel is a circumferential sealing channel.
Embodiment 8: The continuous analyte monitoring device according to any one of the preceding embodiments, wherein the open channel is sealed off from the electronics compartment by a wall of the open channel.
Embodiment 9: The continuous analyte monitoring device according to any one of the preceding embodiments, wherein the input filling port and the output filling port are arranged opposite to each other.
Embodiment 10: The continuous analyte monitoring device according to any one of the preceding embodiments, wherein the input filling port and/or the output filling port are arranged transversely, specifically essentially perpendicularly, to the sealing channel and/or to a direction of extension of the housing.
Embodiment 11: The continuous analyte monitoring device according to any one of the preceding embodiments, wherein the housing further comprises at least one cavity arranged between the sealing channel and the output filling port, wherein the cavity is configured for collecting the sealing material, specifically an excess of the sealing material from the sealing channel, specifically when the sealing material is filled into the sealing channel.
Embodiment 12: The continuous analyte monitoring device according to any one of the preceding embodiments, wherein the housing is at least partially formed as a cylindrical ring, wherein the sealing channel is a circumferential sealing channel, wherein the sealing channel seals the electronics compartment from the sterile compartment or wherein the circumferential sealing channel seals the electronics compartment from an outer environment of the continuous analyte monitoring device.
Embodiment 13: The continuous analyte monitoring device according to any one of the preceding embodiment, wherein the housing comprises two of the sealing channels, wherein the two sealing channels comprise an inner circumferential sealing channel and an outer circumferential sealing channel, wherein the inner circumferential sealing channel has a smaller diameter than the outer circumferential sealing channel.
Embodiment 14: The continuous analyte monitoring device according to the preceding embodiment, wherein each of the inner circumferential sealing channel and an outer circumferential sealing channel respectively comprises one input filling port and one output filling port.
Embodiment 15: The continuous analyte monitoring device according to any one of the two preceding embodiments, wherein inner circumferential sealing channel and the outer circumferential sealing channel and the inner circumferential sealing channel are connected to each other via a connecting channel.
Embodiment 16: The continuous analyte monitoring device according to the preceding embodiment, wherein the housing comprises one input filling port and two output filling ports, wherein the input filling port is arranged at the outer circumferential sealing channel and wherein one of the output filling ports is arranged at the outer circumferential sealing channel and another one of the output filling ports is arranged at the inner circumferential sealing channel.
Embodiment 17: The continuous analyte monitoring device according to any one of the four preceding embodiments, wherein the housing is at least partially formed as a cylindrical ring, wherein the inner circumferential sealing channel seals the electronics compartment from the sterile compartment, wherein the outer circumferential sealing channel seals the electronics compartment from an outer environment of the continuous analyte monitoring device.
Embodiment 18: The continuous analyte monitoring device according to any one of the preceding embodiments, wherein the housing comprises a base plate being configured for attachment to a skin site of a user, specifically via at least one adhesive, and an upper plate, wherein the base plate and the upper plate are configured for forming at least parts of the longitudinal sides of the electronics compartment.
Embodiment 19: The continuous analyte monitoring device according to the preceding embodiment, wherein the lower housing portion is the base plate and wherein the upper housing portion is the upper plate.
Embodiment 20: The continuous analyte monitoring device according to any one of the preceding embodiments, wherein the housing further comprises a connector unit comprising the open channel of the housing, wherein the connector unit has a channel, wherein the analyte sensor passes through the channel.
Embodiment 21: The continuous analyte monitoring device according to the preceding embodiment, wherein the open channel and the channel are arranged transversely, specifically essentially perpendicularly, to each other.
Embodiment 22: The continuous analyte monitoring device according to any one of the two preceding embodiments, wherein the channel is a sealed channel.
Embodiment 23: The continuous analyte monitoring device according to any one of the three preceding embodiments, wherein the channel connects the sterile compartment with the electronics compartment.
Embodiment 24: The continuous analyte monitoring device according to any one of the four preceding embodiments, wherein the analyte sensor comprises an in vivo proximal portion and an ex vivo distal portion, wherein the ex vivo distal portion comprises a first section being received in the channel and a second section being received in the electronics compartment.
Embodiment 25: The continuous analyte monitoring device according to the preceding embodiment, wherein the first section and the second section are essentially arranged along a straight line.
Embodiment 26: The continuous analyte monitoring device according to any one of the two preceding embodiments, wherein the first section and the second section are arranged in an angle of 5° to 90°, specifically of 20° to 80°, more specifically of 40° to 70°, to each other.
Embodiment 27: The continuous analyte monitoring device according to any one of the seven preceding embodiments, wherein the lower housing portion is a lower part of the connector unit and wherein the upper housing portion is an upper part of the connector unit.
Embodiment 28: The continuous analyte monitoring device according to the preceding embodiment, wherein the channel intersects with the sealing channel.
Embodiment 29: The continuous analyte monitoring device according to the preceding embodiment, wherein the channel and the sealing channel are at least partially filled with the sealing material being configured for sealing the channel including the through passing analyte sensor, specifically such that the sterile compartment is sealed off from the electronics compartment.
Embodiment 30: The continuous analyte monitoring device according to the preceding embodiment, wherein the channel is at least partially filled with the sealing material.
Embodiment 31: The continuous analyte monitoring device according to the preceding embodiment, wherein the channel is configured to be filled with the sealing material in an assembled state of the lower part and the upper part.
Embodiment 32: The continuous analyte monitoring device according to any one of the five preceding embodiments, wherein an upper section of the open channel is formed by the upper part of the connector unit, wherein a lower section of the open channel is formed by the lower part of the connector unit.
Embodiment 33: The continuous analyte monitoring device according to any one of the six preceding embodiments, wherein a wall of the open channel is at least partially formed by the lower part of the connector unit and by the upper part of the connector unit.
Embodiment 34: The continuous analyte monitoring device according to any one of the six preceding embodiments, wherein at least one of: a width of a lumen of the sealing channel; a shape of the sealing channel, specifically a shape of a cross-section of the sealing channel; a dimension of the sealing channel, specifically a dimension of the cross-section of the sealing channel; varies along the sealing channel.
Embodiment 35: The continuous analyte monitoring device according to the preceding embodiment, wherein at least one of: the width of the lumen of the sealing channel; the dimension of the sealing channel, specifically the dimension of the cross-section of the sealing channel; a cross-sectional area of the sealing channel, specifically a cross-sectional area of the sealing channel transverse to the direction of extension of the sealing channel; may decrease, specifically continuously, the further a section of the sealing channel is away from the channel.
Embodiment 36: The continuous analyte monitoring device according to any one of the two preceding embodiments, wherein the sealing channel is divided into a first arm and into a second arm by the input filling port and by the output filling port, wherein the first arm intersects with the channel, wherein at least one of: the width of the lumen of the sealing channel; a mean width of the lumen of the sealing channel; the dimension of the sealing channel, specifically the dimension of the cross-section of the sealing channel; a mean dimension of the sealing channel, specifically a mean dimension of the cross-section of the sealing channel; a cross-sectional area of the sealing channel, specifically the cross-sectional area of the sealing channel transverse to the direction of extension of the sealing channel; a mean cross-sectional area of the sealing channel, specifically a mean cross-sectional area of the sealing channel transverse to the direction of extension of the sealing channel; may be larger in the first arm than in the second arm.
Embodiment 37: The continuous analyte monitoring device according to any one of the preceding embodiments, wherein the housing comprises an upper side and a lower side, wherein the open channel connects the upper side and the lower side.
Embodiment 38: The continuous analyte monitoring device according to the preceding embodiment, wherein the removable sterility cap is located on the lower side of the housing and wherein at least a part of the insertion cannula holder extends from the upper side of the housing.
Embodiment 39: The continuous analyte monitoring device according to any one of the two preceding embodiments, wherein the insertable portion of the analyte sensor extends downwardly inside the open channel beyond the lower side.
Embodiment 40: The continuous analyte monitoring device according to any one of the preceding embodiments, wherein the insertion cannula holder and the removable sterility cap are respectively removably connected to the housing, specifically on opposing sides of the housing, specifically of the open channel.
Embodiment 41: The continuous analyte monitoring device according to any one of the preceding embodiments, wherein the open channel at least partially circumferentially surrounds the analyte sensor and the insertion cannula.
Embodiment 42: The continuous analyte monitoring device according to any one of the preceding embodiments, wherein the analyte sensor comprises at least two electrodes each comprising an electrode conductor path configured for transmitting a sensor current for detecting the analyte.
Embodiment 43: The continuous analyte monitoring device according to the preceding embodiment, wherein the analyte sensor comprises a carrier, specifically a substrate, wherein the at least two electrode conductor paths are disposed on the carrier.
Embodiment 44: The continuous analyte monitoring device according to any one of the two preceding embodiments, wherein the at least two electrodes are a working electrode configured for detecting the analyte and a further electrode, wherein the further electrode is selected from the group consisting of: a counter electrode, a reference electrode, and a combined counter-reference electrode.
Embodiment 45: The continuous analyte monitoring device according to any one of the preceding embodiments, wherein the electronics unit comprises a printed circuit board.
Embodiment 46: The continuous analyte monitoring device according to any one of the preceding embodiments, wherein the insertion cannula is fixedly attached to the insertion cannula holder.
Embodiment 47: The continuous analyte monitoring device according to any one of the preceding embodiments, wherein the insertion cannula holder is configured for removal after insertion of the insertable portion of the analyte sensor into the body tissue.
Embodiment 48: The continuous analyte monitoring device according to any one of the preceding embodiments, wherein the insertion cannula holder at least partially surrounds the insertion cannula.
Embodiment 49: The continuous analyte monitoring device according to any one of the preceding embodiments, wherein the insertion cannula holder forms a removable cap, specifically a removable upper cap, configured for sealing an end of the open channel of the housing.
Embodiment 50: The continuous analyte monitoring device according to any one of the preceding embodiments, wherein the insertion cannula holder is at least partially received inside the open channel.
Embodiment 51: The continuous analyte monitoring device according to any one of the preceding embodiments, wherein the insertion cannula holder is removably connected to the housing and/or to the removable sterility cap via a connection, specifically via at least one of a force fit connection, a form fit connection, a screwing connection, a magnetic connection or a bayonet connection.
Embodiment 52: The continuous analyte monitoring device according to any one of the preceding embodiments, wherein the removable sterility cap is removably connected to the housing and/or to the insertion cannula holder via a connection, specifically via at least one of a force fit connection, a form fit connection, a screwing connection, a magnetic connection or a bayonet connection.
Embodiment 53: The continuous analyte monitoring device according to any one of the preceding embodiments, wherein the removable sterility cap is configured for removal before insertion of the insertable portion of the analyte sensor into the body tissue.
Embodiment 54: The continuous analyte monitoring device according to any one of the preceding embodiments, wherein the continuous analyte monitoring device further comprises an insertion device.
Embodiment 55: The continuous analyte monitoring device according to the preceding embodiment, wherein the insertion device comprises an insertion mechanism.
Embodiment 56: The continuous analyte monitoring device according to any one of the preceding embodiments, wherein the continuous analyte monitoring device is a disposable medical device.
Embodiment 57: The continuous analyte monitoring device according to any one of the preceding embodiments, wherein the electronics unit is a single-use electronics unit.
Embodiment 58: The continuous analyte monitoring device according to any one of the preceding embodiments, wherein the continuous analyte monitoring device forms a preassembled single unit.
Embodiment 59: The continuous analyte monitoring device according to any one of the preceding embodiments, wherein the sealing material is configured for fixedly attaching the lower housing portion and the upper housing portion to each other.
Embodiment 60: A continuous analyte monitoring system, wherein the continuous analyte monitoring system comprises:
a continuous analyte monitoring device according to any one of the preceding embodiments;
an insertion device at least partially covering the continuous analyte monitoring device, wherein the insertion device is configured for enabling a user to drive the insertion cannula into the body tissue and to insert the insertable portion of the analyte sensor.
Embodiment 61: The continuous analyte monitoring system according to the preceding embodiment, wherein the continuous analyte monitoring system further comprises:
a sensor controller which is coupled to the analyte sensor, wherein the sensor controller is configured to receive analyte sensor data from the analyte sensor; and
a remote control which is configured to receive sensor data from the sensor controller and to process and/or display sensor data.
Embodiment 62: A method of assembling the analyte monitoring device according to any one of the preceding embodiments referring to an analyte monitoring device, wherein the method comprises:
a) Providing the lower housing portion;
b) Mounting the analyte sensor onto the lower housing portion;
c) Placing the upper housing portion onto the lower housing portion;
d) Injecting the sealing material into the sealing channel via the input filling port, specifically under pressure, such that the lower housing portion and the upper housing portion are fixedly attached to each other and such that the interior space enclosed by the upper housing portion and the lower housing portion is sealed.
Embodiment 63: Method of using a continuous analyte monitoring device according to any one of the preceding embodiments referring to a continuous analyte monitoring device, the method comprising:
I. providing the continuous analyte monitoring device;
II. removing the removable sterility cap;
III. inserting the analyte sensor into a body tissue of the user; and
IV. removing the insertion cannula holder, thereby removing the insertion cannula from the continuous analyte monitoring device.

### BRIEF DESCRIPTION OF THE DRAWINGS

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of this disclosure is not restricted by the preferred embodiments. The embodiments are schematically depicted in the Figures. Therein, identical reference numbers in these Figures refer to identical or functionally comparable elements.

The above-mentioned aspects of exemplary embodiments will become more apparent and will be better understood by reference to the following description of the embodiments taken in conjunction with the accompanying drawings, wherein:

FIGS. 1A and 1B show an exemplary embodiment of a continuous analyte monitoring device according to this disclosure in different cross-sectional views;

FIGS. 2A to 2E show an exemplary embodiment of components of a continuous analyte monitoring device according to this disclosure in different cross-sectional views;

FIGS. 3A to 3C show an exemplary embodiment of components of a continuous analyte monitoring device according to this disclosure in different cross-sectional views;

FIG. 4 shows an exemplary embodiment of a continuous analyte monitoring device according to this disclosure in a cross-sectional view;

FIGS. 5A to 5B show an exemplary embodiment of components of a continuous analyte monitoring device according to this disclosure in a perspective view and in a top view;

FIG. 6 shows an exemplary embodiment of components of a continuous analyte monitoring device according to this disclosure in a cross-sectional view;

FIG. 7 shows an exemplary embodiment of components of a continuous analyte monitoring device according to this disclosure in a cross-sectional view; and

FIG. 8 shows an exemplary embodiment of a continuous analyte monitoring system according to this disclosure in a schematic view.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

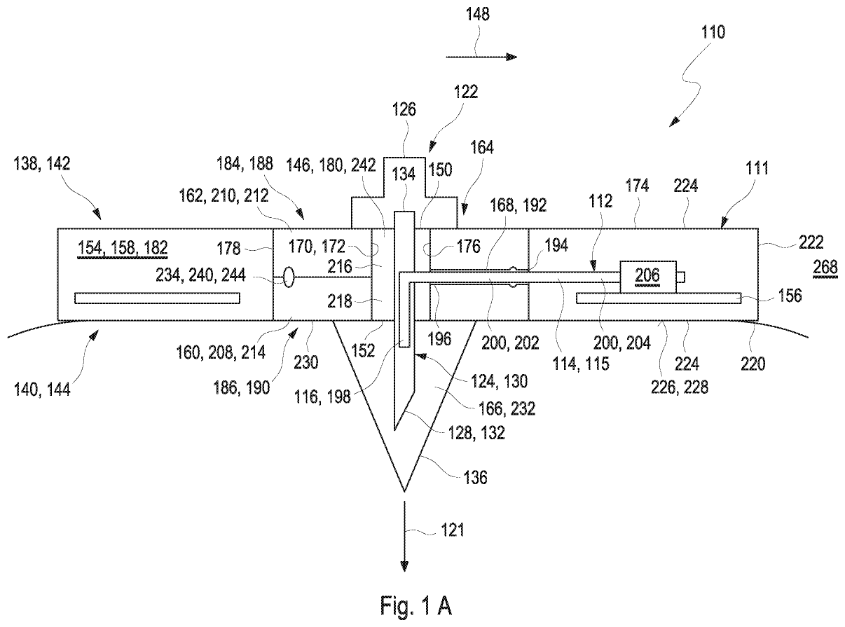

FIGS. 1A and 1B show an exemplary embodiment of a continuous analyte monitoring device 110 according to this disclosure in different cross-sectional views. FIG. 1B shows a cross-sectional view along a plane extending parallel to a direction of extension of a housing 111 of the continuous analyte monitoring device 110. FIG. 1A shows a cross-sectional view of the continuous analyte monitoring device 110 according to FIG. 1B (section A-A, see FIG. 1B).

The continuous analyte monitoring device 110 comprises an analyte sensor 112. The analyte sensor 112 is configured for detecting an analyte in a body fluid of a user.

The analyte sensor 112 may comprise a carrier 114, specifically a substrate 116. At least two electrodes may be disposed on the carrier 114. The carrier 114, specifically the substrate 116, specifically may have an elongated shape, such as a strip-shape and/or a bar-shape.

The analyte sensor 112 has an insertable portion 116 adapted for at least partially being inserted into a body tissue of a use. The insertable portion 116 may be called in-vivo proximal portion 198. A portion of the analyte sensor 112 which may stay outside of the body tissue may also be called the ex vivo distal portion 200. The ex vivo distal portion 200 and the in vivo proximal portion 198 may be arranged transversely, specifically essentially perpendicularly, to each other. The in vivo proximal portion 198 may extend along a direction of insertion 121. The direction of insertion 121 may be transverse, specifically essentially perpendicular, to a skin site of the user.

Further, the continuous analyte monitoring device 110 comprises an insertion component 122 comprising an insertion cannula 124 and an insertion cannula holder 126.

The analyte sensor 112 is at least partially placed inside the insertion cannula 124. The insertion cannula 124 may comprise a tip or a sharp end 128 for inserting the analyte sensor 112 at least partially into the body tissue. Specifically, the insertion cannula 124 may be a slotted cannula 130. The insertion cannula 124 may be configured to be inserted essentially vertically relative to the body tissue of the user. The insertion cannula holder 126 may be configured for removal after insertion of the insertable portion 116 of the analyte sensor 112 into the body tissue.

The insertion cannula 124 is attached to the insertion cannula holder 126. Specifically, the insertion cannula 124 may be fixedly attached to the insertion cannula holder 126. The insertion cannula holder 126 may at least partially surround the insertion cannula 124. Specifically, the insertion cannula 124 may have a first end 132 and an opposing second end 134. The first end 132 may be the sharp end 128 for inserting the analyte sensor 112 at least partially into the body tissue. The second end 134 may be attached to the insertion cannula holder 126.

Further, the continuous analyte monitoring device 110 comprises a removable sterility cap 136. The removable sterility cap 136 at least partially surrounds the insertable portion 116 of the analyte sensor 112.

The removable sterility 136 cap at least partially surrounds the insertable portion 116 of the analyte sensor 112. Thus, the insertable portion 116 may be at least partially received in the removable sterility cap 136. The removable sterility cap 136 may be configured for removal before insertion of the insertable portion 116 of the analyte sensor 112 into the body tissue.

Further, the continuous analyte monitoring device 110 comprises the housing 111.

The housing 111 may comprise an upper side 138 and a lower side 140. Specifically, the upper side 138 may refer to a distal side 142 of the housing 111. Specifically, the lower side 140 may refer to a proximal side 144 of the housing 111. The housing 111 comprises an open channel 146 which at least partially surrounds the analyte sensor 112 and the insertion component 122. The open channel 146 may extend transversely, specifically essentially perpendicularly, relative to the lower side 140 and to the upper side 138 of the housing 111.

The open channel 146 may connect the upper side 138 and the lower side 140. The open channel 146 may specifically be an essentially straight channel. Further, the open channel 146 may extend transversely, specifically essentially perpendicularly, to a direction of extension 148 of the housing 111. The open channel 146 may specifically have an upper side opening 150 and an opposing lower side opening 152. The upper side opening 150 may be located on the upper side 138 of the housing 111 facing away from the skin site and the lower side opening 152 may be located on the lower side 140 of housing 111 facing the skin site.

Specifically, the open channel 146 may at least partially circumferentially surround the analyte sensor 112 and the insertion component 122, specifically at least the insertion cannula 124 of the insertion component 122. The housing 111 may be at least partially formed as a cylindrical ring at least partially surrounding the analyte sensor 112 and the insertion component 122, specifically at least the insertion cannula 124 of the insertion component 122 and. The insertable portion 116 of the analyte sensor 112 may extend downwardly inside the open channel 146 beyond the lower side of 140 the housing 111.

The housing 111 further comprises an electronics compartment 154 with an electronics unit 156 received therein. The electronics compartment 154 may be a sealed compartment 158. The electronics unit 156 may be fixedly positioned within the electronics compartment 154 of the housing 111.

The housing 111 is at least partially formed by a lower housing portion 160 and an upper housing portion 162.

The housing 111 may further comprise a connector unit 164 comprising the open channel 146 of the housing 111. Specifically, the connector unit 164 may be configured for connecting a sterile compartment 166 with the electronics compartment 154. Specifically, the connector unit 164 may have a channel 192 and the analyte sensor 112 may pass through the channel 192. The channel 192 may also be referred to as opening 168. The analyte sensor 112 may be partially received in the sterile compartment 166, specifically inside the open channel 146, and partially received in the electronics compartment 154. More specifically, the analyte sensor 112 may be partially received in the sterile compartment 166, specifically inside the open channel 146, partially received within the channel 192 of the connector unit 164 and partially received in the electronics compartment 154.

The open channel 146 may be sealed off from the electronics compartment 154 by a wall 170 of the open channel 146. The wall 170 of the open channel 146 may be formed by the connector unit 164. Specifically, the connector unit 164 may have a connector unit wall 172, specifically extending in the direction of insertion 121 of the analyte sensor 112 or extending transversely, specifically perpendicularly, to longitudinal sides 174 of the housing 111. The connector unit wall 172 may have a first side 176 and an opposing second side 178. The first side 176 may face an interior space 180 of the open channel 146. Further, second side 178 may face an interior space 182 of the electronics compartment 154.

The connector unit 164 may have an upper connector unit side 184 and a lower connector unit side 186. The open channel 146 may connect the upper connector unit side 184 and the lower connector unit side 186. Specifically, the upper connector unit side 184 may refer to a distal connector unit side 188 of the connector unit 164. Specifically, the lower connector unit side 186 may refer to a proximal connector unit side 190 of the connector unit 164.

The open channel 146 and the channel 192 may be arranged transversely, specifically essentially perpendicularly, to each other. The channel 192 may be formed by a hole or by a cutout within the connector unit 164, specifically in the wall 170 of the connector unit 164. The channel 192 itself may be embodied an essentially straight channel. The channel 192 may have a first end 194 and an opposing second end 196. The channel 192 may connect the sterile compartment 166 with the electronics compartment 154 via the first end 194 and the second end 196.

The ex vivo distal portion 200 and the in vivo proximal portion 198 of the analyte sensor 112 may be arranged transversely, specifically essentially perpendicularly, to each other. The in vivo proximal portion 198 may extend along the direction of insertion 121. The ex vivo distal portion 200 may comprise a first section 202 being received in the channel 192 and a second section 204 being received in the electronics compartment 154. The electronics unit 156 may be electrically connected to the analyte sensor 112. Thus, specifically, the second section 204 of the ex vivo distal portion 200 may be electrically connected to an electronics component 206 of the electronics unit 156.

The connector unit 164 may specifically comprise a lower part 208 and an upper part 210. The lower part 208 and the upper part 210, in conjunction may form the connector unit 164. Specifically, the upper part 210 may refer to a distal part 212 of the connector unit 164. Specifically, the lower part 208 may refer to a proximal part 214 of the connector unit 164. An upper section 216 of the open channel 146 may be formed by the upper part 210 of the connector unit 164 and a lower section 218 of the open channel 146 may be formed by the lower part 208 of the connector unit 164. Further, the wall 170 of the open channel 146 may be at least partially formed by the lower part 208 of the connector unit 164 and by the upper part 210 of the connector unit 164.

The housing 111 may comprise a base plate 220 being configured for attachment to a skin site of a user, specifically via at least one adhesive. Further, the housing 111 may comprise an upper plate 222. The base plate 220 and the upper plate 222 are configured for forming at least parts of the longitudinal sides 224 of the electronics compartment 154. Specifically, the base plate 220 may comprise a lower surface 226 configured for being placed on a user's skin. The lower surface 226 may exemplarily have a shape of a circular ring surrounding the analyte sensor 112. More specifically, the lower surface 226 may be or may comprise an adhesive surface 228 for attachment to the user's skin. Specifically, the base plate 220 and the upper plate 222, in conjunction may form an encapsulation for the electronics unit 156. Specifically, the electronics compartment 154 may be formed by the base plate 220, the upper plate 222 and the connector unit 164. The longitudinal sides 224 of the electronics compartment 154 may be formed by the base plate 220 and the upper plate 222 and, optionally, also by longitudinal sides 230 of the connector unit 164. The lower side 140 of the housing 111 may be formed by the base plate 220 and by the lower connector unit side 186 of the connector unit 164. The upper side 138 of the housing 111 may be formed by the upper plate 222 and by the upper connector unit side 184 of the connector unit 164.

The removable sterility cap 136, the open channel 146 of the housing 111 and the insertion cannula holder from the sterile compartment 166 for the insertion cannula 124 and at least the insertable portion 116 of the analyte sensor 112. Thus, the insertion cannula 124 and at least the insertable portion 116 of the analyte sensor 112 may be received in the sterile compartment 166. The sterile compartment 166 may also be referred to as sensor compartment 232.

As outlined above, the housing 111 may comprise the upper side 138 and the lower side 140 and the open channel 146 may connect the upper side 138 and the lower side 140. The removable sterility cap 136 may be at least partially located on the lower side 140 of the housing 111, specifically of the connector unit 164, and at least a part of the insertion cannula holder 126 may extend from the upper side 138 of the housing 111, specifically of the connector unit 164. The insertion cannula holder 126 and the removable sterility cap 136 may be respectively removably connected to the housing 111, specifically to the connector unit 164, specifically on opposing sides of the housing 111, specifically of the connector unit 164, more specifically of the open channel 146. The insertion cannula holder 126 may seal with the upper side opening 150 of the open channel 146 and the removable sterility cap 136 may seal with the lower side opening 152 of the open channel 146.

The housing 111 comprises a sealing channel 234 comprising an input filling port 236 and an output filling port 238. The sealing channel 234 is at least partially filled with a sealing material 240 being configured for fixedly attaching the lower housing portion 160 and the upper housing portion 162 to each other and for sealing an interior space 242 enclosed by the upper housing portion 162 and the lower housing portion 160.

In the embodiment according to FIGS. 1A and 1B, the lower housing portion 160 may be the lower part 208 of the connector unit 164 and the upper housing portion 162 may by the upper part 210 of the connector unit 164. Specifically, the sealing channel 234 may be a circumferential sealing channel 244 and the sealing channel 234 may seal the electronics compartment 154 from the sterile compartment 166. The sealing channel 234 may be partially formed by the lower part 208 of the connector unit 164 and may be partially formed by the upper part 210 of the connector unit 164. The sealing channel 234 may be located between the lower part 208 of the connector unit 164 and the upper part 210 of the connector unit 164. The sealing channel 234 may surround, specifically circumferentially surround the open channel 146 of the housing 111.

Specifically, the channel 192 may intersect the sealing channel 234. The channel 192 may be fluidically connected to the sealing channel 234 through the intersection of the sealing channel 234 via the channel 192. Specifically, the channel 192 may intersect the sealing channel 234 between the first end 194 of the channel 192 and the second end 196 of the channel 192. The sealing channel 234 may be arranged between the first end 194 of the channel 192 and the second end 196 of the channel 192. The channel 192 may be at least partially filled with the sealing material 240.

Specifically, the sealing channel 234 and the channel 192 are configured to be filled with the sealing material in an assembled state of the lower part 208 and the upper part 210. Thus, by filling the sealing channel 234 via the input filling port 236, the channel 192 may be sealed with the sealing material 240 as well. Specifically, in an assembled state and manufactured state of the continuous analyte monitoring device 110, the channel 192 may be at least partially filled, preferably fully filled with the sealing material 240.

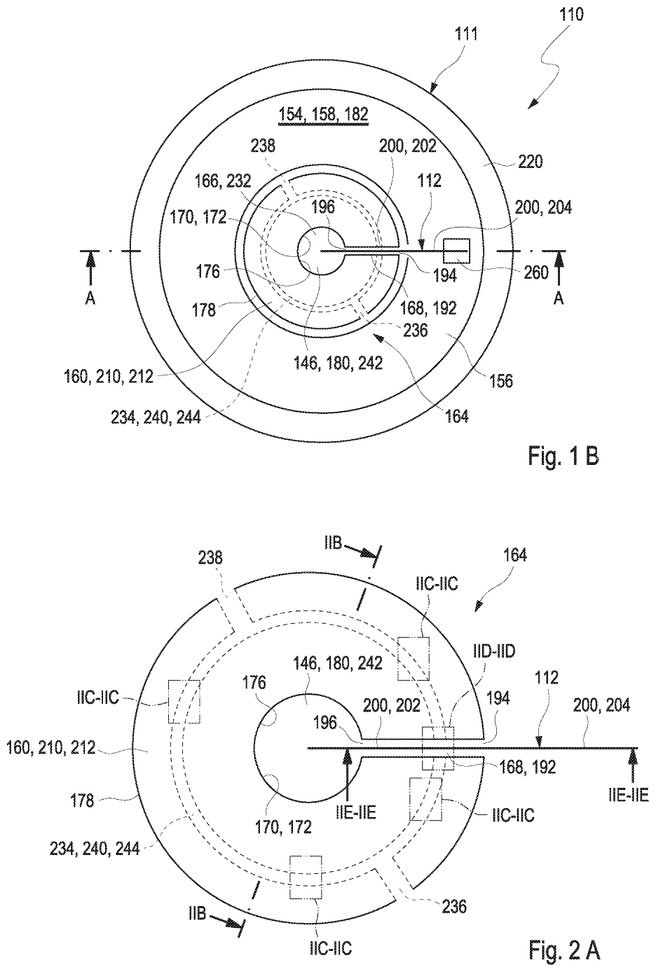

FIGS. 2A to 2E show an exemplary embodiment of components of a continuous analyte monitoring device 110 according to this disclosure in different cross-sectional views. The cross-sectional view according to FIG. 2A corresponds to the cross-sectional view according to FIG. 1B. However, the electronics compartment 154 with the electronics unit 156 received therein is not illustrated. Thus, reference is made to the description of FIGS. 1A and 1B above.

FIG. 2B shows a cross-sectional view of components of the continuous analyte monitoring device 110 according to FIG. 2A (section A-A, see FIG. 2A). Thereby, the upper part 210 and the lower part 208 of the connector unit 164 are shown in a disassembled state (Figures above) and in an assembled state (Figures below).

As can be seen in FIG. 2B, the sealing channel 234 may be partially formed by a groove 246 within the lower part 208 of the connector unit 164, specifically within a surface 248 of the lower part 208 of the connector unit 164. The upper part 210 of the connector unit 164 may form a cover 250 for the groove 246 within the lower part 208 of the connector unit 164. Thus, the sealing channel 234 may be partially formed by the groove 246 within lower part 208 of the connector unit 164 and by a flat surface 252 of upper part 210 of the connector unit 164.

Specifically, the sealing channel 234 may be configured to be filled with the sealing material 240 in an assembled state of the lower part 208 and the upper part 210. Thus, before the lower part 208 and the upper part 210 are assembled such as illustrated in the upper illustration of FIG. 2B, the sealing channel 234 may be free from the sealing material 240. In the assembled state of the lower part 208 and the upper part 210 such as illustrated in the lower illustration of FIG. 2B, the sealing material 240 may be filled into the sealing channel 234 via the input filling port 236.

The sealing channel 234 may sealingly connect the lower part 208 and the upper part 210 of the connector unit 164 such that the sterile compartment 166 may be sealingly isolated from the electronics compartment 154. Further, the sealing channel 234 may seal the analyte sensor 112 passing through the channel 192. To accomplish the sealing, it may be envisioned to mount the analyte sensor 112 onto the lower part 208 of the connector unit 164 followed by attaching the upper part 210 of the connector unit 164. In this state, the channel 192 and the sealing channel 234 are not yet sealed. By injecting the sealing material 240 into the input filling port 236 under pressure, the sealing material 240 may fill the channel 192 and the sealing channel 234 and seal analyte sensor 112 within the channel 192. This way an effective sealing can be accomplished.

FIG. 2C shows a cross-sectional view of components of the continuous analyte monitoring device 110 according to FIG. 2A (section B-B, see FIG. 2A). Thereby, the upper part 210 and the lower part 208 of the connector unit 164 are shown in an assembled state.

As can be seen in FIG. 2C, the cross-section of the sealing channel 234 may vary along the sealing channel 234. Thus, as illustrated in FIG. 2C, in certain areas, the sealing channel 234 may be formed by the groove 246 within the lower part 208 of the connector unit 164, specifically within the surface 248 of the lower part 208 of the connector unit 164, and by a groove 254 within the upper part 210 of the connector unit 164, specifically within a surface 256 of the upper part 210 of the connector unit 164.

FIG. 2D shows a cross-sectional view of components of the continuous analyte monitoring device 110 according to FIG. 2A (section C-C, see FIG. 2A). Thereby, the upper part 210 and the lower part 208 of the connector unit 164 are shown in an assembled state. FIG. 2E shows a cross-sectional view of components of the continuous analyte monitoring device 110 according to FIG. 2A (section D-D, see FIG. 2A). Thereby, the upper part 210 and the lower part 208 of the connector unit 164 are shown in an assembled state. Further, in FIG. 2E, the analyte sensor 112 which passes through the channel 192 is illustrated.

As illustrated in FIGS. 2D and 2E, the channel 192 through which the analyte sensor 112 passes may intersect the sealing channel 234. The channel 192 may be at least partially filled with the sealing material 240. The sealing material 240 may circumferentially surround the analyte sensor 112, specifically the section of the analyte sensor 112 which passes through the channel 192.

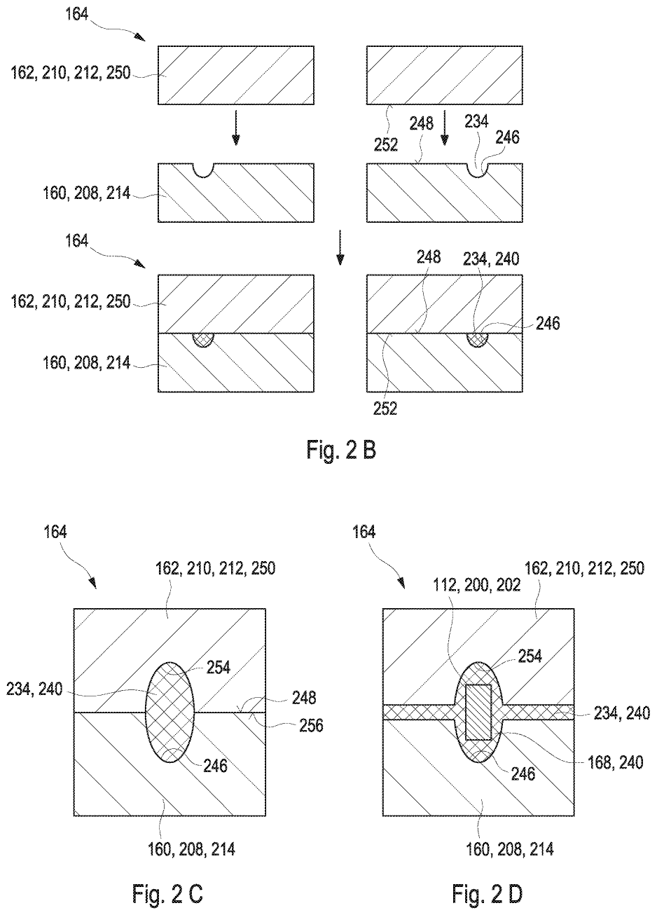

FIGS. 3A to 3C show an exemplary embodiment of components of a continuous analyte monitoring device 110 according to this disclosure in different cross-sectional views. The cross-sectional view according to FIG. 3A corresponds at least partially to the cross-sectional view according to FIG. 2A. Thus, reference is made to the description of FIGS. 2A and 2E above.

FIG. 3B shows a cross-sectional view of components of the continuous analyte monitoring device 110 according to FIG. 3A (sections B-B, see FIG. 3A). Thereby, the upper part 210 and the lower part 208 of the connector unit 164 are shown in an assembled state. Further, FIG. 3C shows a cross-sectional view of components of the continuous analyte monitoring device 110 according to FIG. 3A (sections E-E, see FIG. 3A). Thereby, the upper part 210 and the lower part 208 of the connector unit 164 are shown in an assembled state.

As can be seen the FIGS. 3B and 3C, a width of a lumen 258 of the sealing channel 234 in an area close to the sealing channel 234 intersecting with the channel 192 may be larger, such as illustrated in FIG. 3C, than in an area being further away from the sealing channel 234 intersecting with the channel 192 such as illustrated in FIG. 3B. Further, a cross-sectional area of the sealing channel 234 may be larger in an area close to the sealing channel 234 intersecting with the channel 192 than in an area being further away from the sealing channel 234 intersecting with the channel 192. Specifically, the sealing channel 234 may be divided into a first arm 257 and into a second arm 259 by the input filling port 236 and by the output filling port 238. The first arm 257 may intersect with the channel 192. The width of the lumen 258 of the sealing channel 234, specifically a mean width of the lumen 258 of the sealing channel 234, may be larger in the first arm 257 than in the second arm 259.

At the intersection of the channel 192 with the sealing channel 234, the analyte sensor 112 may impose an increased mechanical resistance to a flow of the sealing material 240. To avoid that the sealing material 240 only flows along an arm of the sealing channel 234 in the area being further away from the sealing channel, the lumen 258 of the sealing channel 234 may increase such that differences in flow resistance are balanced out.

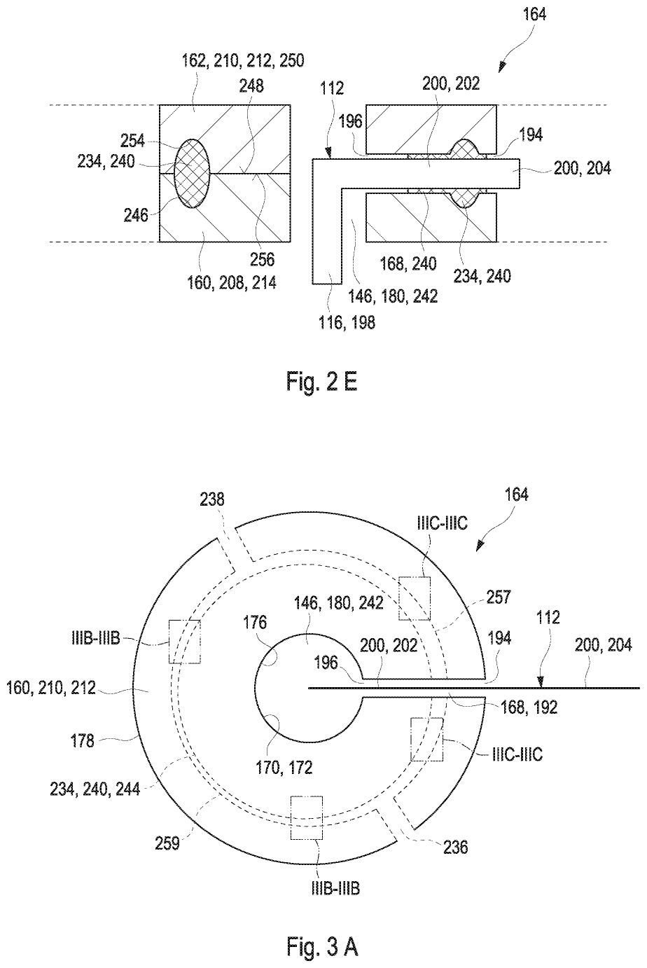

FIG. 4 shows an exemplary embodiment of a continuous analyte monitoring device 110 according to this disclosure in a cross-sectional view. The cross-sectional view according to FIG. 2A at least partially corresponds to the cross-sectional view according to FIG. 1B. Thus, reference is made to the description of FIGS. 1A and 1B above.

In contrast to the embodiment according to FIGS. 1A and 1B, wherein the first section 202 and the second section 204 of the ex vivo distal portion 200 may be essentially arranged along a straight line, the first section 202 and the second section 204 of the ex vivo distal portion 200 may be arranged in an angle of 40° to 70°, specifically of 55° to 65°, to each other such as illustrated in FIG. 4. Thereby, a positioning and stabilization of the analyte sensor 112 within the connector unit 164 may be improved.

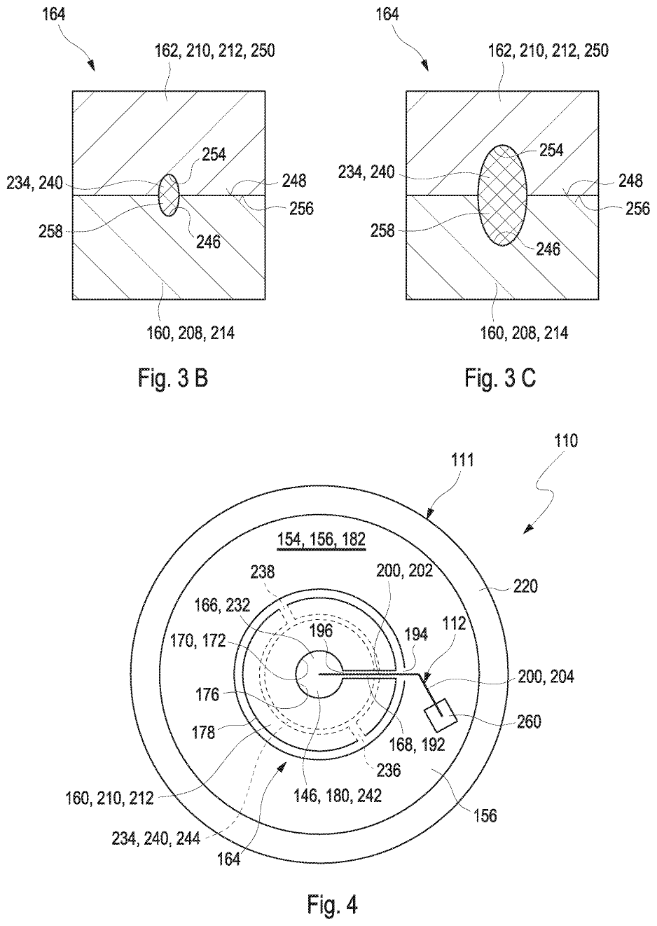

FIGS. 5A to 5B show an exemplary embodiment of components of a continuous analyte monitoring device 110 according to this disclosure. FIG. 5A shows an exemplary embodiment of a housing 111 of the continuous analyte monitoring device 110 in a perspective view. FIG. 5B shows a top view on a base plate 220 of the housing 111 of the continuous analyte monitoring device 110. The housing 111 of the continuous analyte monitoring device 110 as illustrated in FIGS. 5A and 5B may at least partially correspond to the continuous analyte monitoring device 110 as illustrated in FIGS. 1A and 1B. Thus, reference is made to the description of FIGS. 1A and 1B above.

In the embodiment according to FIGS. 5A and 5B, the lower part 208 of the connector unit 164 and the base plate 220 may specifically form a single piece. The lower housing portion 160 may be the base plate 220 and the upper housing portion 162 may be the upper plate 222, specifically in conjunction with the upper part 210.

Specifically, the housing 111 may comprise two of the sealing channels 234. The two sealing channels 234 may comprise an inner circumferential sealing channel 264 and an outer circumferential sealing channel 266. The inner circumferential sealing channel 264 may have a smaller diameter than the outer circumferential sealing channel 266.

The inner circumferential sealing channel 264 may seal the electronics compartment 154 from the sterile compartment 166. The inner circumferential sealing channel 264 may surround, specifically circumferentially surround the open channel 146 of the housing 111. The inner circumferential sealing channel 264 may be arranged between the sterile compartment 166, specifically the open channel 146, and the electronics compartment 154.

The outer circumferential sealing channel 266 may seal the electronics compartment 154 from an outer environment 268 of the continuous analyte monitoring device 110. The outer circumferential sealing channel 266 may surround, specifically circumferentially surround the electronics unit 156 received in the housing 111. The outer circumferential sealing channel 266 may be located in a wall 270 separating the electronics compartment 154 from the outer environment 268 of the continuous analyte monitoring device 110.

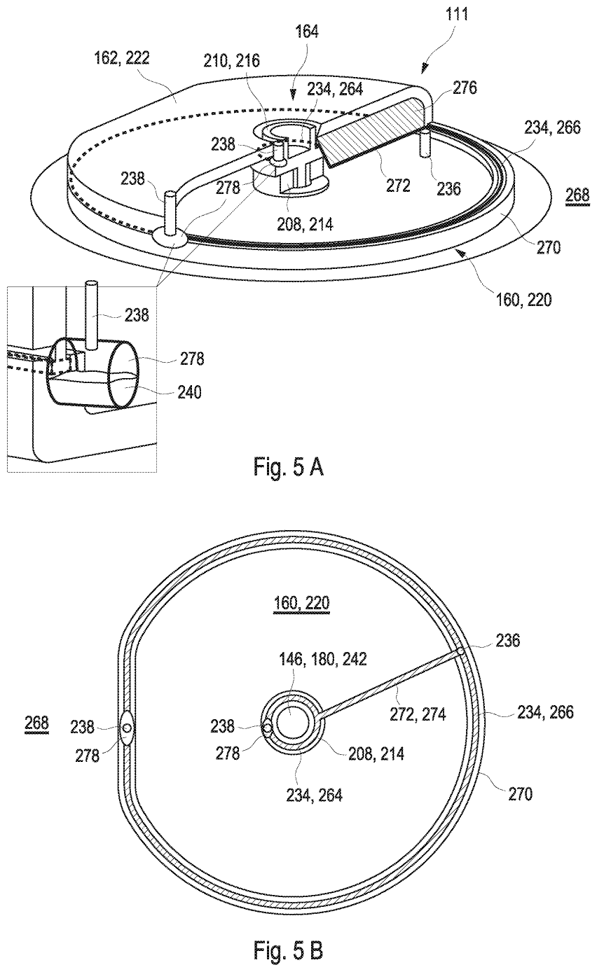

Further, specifically, the inner circumferential sealing channel 264 and the outer circumferential sealing channel 266 may be connected to each other, specifically via a connecting channel 272. The connecting channel 272 specifically be may configured for fluidically connecting the inner circumferential sealing channel 264 and the outer circumferential sealing channel 266. The sealing material 240 may be transferred from one of the inner circumferential sealing channel 264 and the outer circumferential sealing channel 266 to the other one of the inner circumferential sealing channel 264 and the outer circumferential sealing channel 266 the connecting channel 272. The connecting channel 272 may be embodied as an essential straight channel. The connecting channel 272 may specifically be formed within the lower housing portion 160, specifically within the base plate 220. Specifically, the connecting channel 272 may be formed as a groove 274 within the lower housing portion 160. The groove 274 may be located on a side of the lower housing portion 160 facing the electronics compartment 154. The groove 274 may be covered with a cover 276, specifically such that the connecting channel 274 is formed (see FIG. 6).

The housing 111 may comprise one input filling port 236 and two output filling ports 238. Specifically, the input filing port 236 and the output filling ports 238 may extend transversely, specifically perpendicularly, to a plane along which the sealing channel 234 extends. The input filling port 236 and the output filling ports 238 may be accessible from the outer environment 268 of the continuous analyte monitoring device 110.

Specifically, the input filling port 236 may be arranged at the outer circumferential sealing channel 266. The input filling port 236 may be fluidically connected to the outer circumferential sealing channel 266. Further, specifically, one of the two output filling ports 238 may be arranged at the outer circumferential sealing channel 266 and another one of the two output filling ports 238 may be arranged at the inner circumferential sealing channel 264. Specifically, one of the two output filling ports 238 may be fluidically connected to the outer circumferential sealing channel 266 and another one of the two output filling ports 238 may be fluidically connected to the inner circumferential sealing channel 264.

Further, the housing 111 may further comprise a cavities 278 arranged between the sealing channel 234 and the output filling ports 238. Specifically, the cavities 278 may be fluidically arranged between the sealing channel 234 and the output filling ports 238. The cavities 278 may provide a free volume. The cavities 278 may be configured for collecting the sealing material 240.

FIG. 6 shows an exemplary embodiment of components of a continuous analyte monitoring device 110 according to this disclosure in a cross-sectional view. The components of the continuous analyte monitoring device 110 as illustrated in FIG. 6 may at least partially correspond to the components of the continuous analyte monitoring device 110 as illustrated in FIGS. 5A and 5B. Thus, reference is made to the description of FIGS. 5A and 5B above.

During injecting the sealing material 240 into the sealing channel 234 via the input filling port 236, the assembly of the lower housing portion 160 and the upper housing portion 162 may be tilted. The housing 111 may comprise the cavities 278 arranged between the sealing channel 234 and the output filling ports 238. Specifically, the an assembly of the lower housing portion 160 and the upper housing portion 162 may be continuously tilted such that the cavities 278 continuously represent a highest point at an end of a flow front of the sealing material 240 through the sealing channel 234. Thus, air may escape from the sealing channel 234, specifically via the output filling ports 238. By reducing a probability of air entering or staying within the sealing channel 234, a tightness of a bonded joint between the lower housing portion 160 and the upper housing portion 162 may be more reliable and production waste may be reduced.

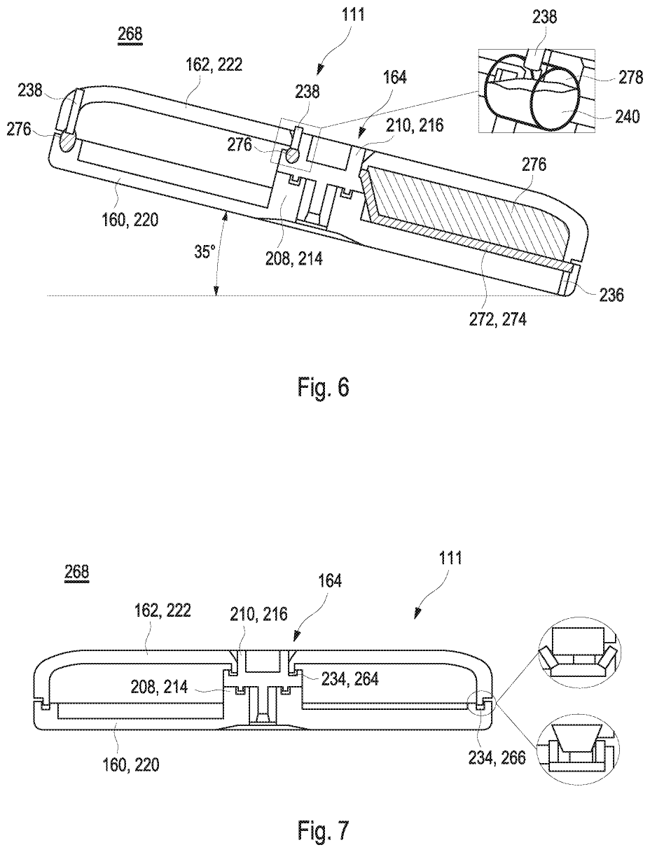

FIG. 7 shows an exemplary embodiment of components of a continuous analyte monitoring device 110 according to this disclosure in a cross-sectional view. The components of the continuous analyte monitoring device 110 as illustrated in FIG. 7 may at least partially correspond to the components of the continuous analyte monitoring device 110 as illustrated in FIGS. 5A and 5B. Thus, reference is made to the description of FIGS. 5A and 5B above. As can specifically be seen in the detailed pictures of FIG. 7, the sealing channel may be realized via geometrical shapes such as receptacles and protrusions within the lower housing portion 160 and the upper housing portion 162.

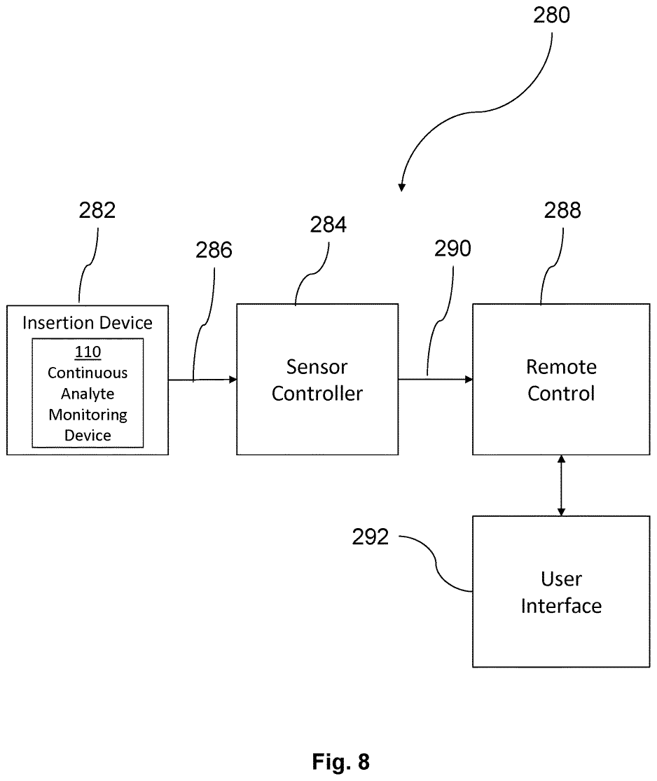

FIG. 8 shows an exemplary embodiment of a continuous analyte monitoring system 280 according to this disclosure in a schematic view.

The continuous analyte monitoring system 280 comprises a continuous analyte monitoring device 110. The continuous analyte monitoring device 110 may correspond to the embodiments such as illustrated in FIGS. 1A and 1B. Thus, reference is made to the description of these figures above.

The continuous analyte monitoring system 280 further comprises an insertion device 282 at least partially covering the continuous analyte monitoring device 110, wherein the insertion device 282 is configured for enabling a user to drive the insertion cannula 124 into the body tissue and to insert the insertable portion 116 of the analyte sensor 112.

The continuous analyte monitoring system 280 may further comprise a sensor controller 284 which may be coupled to the analyte sensor 112 of the continuous analyte monitoring device 110. The sensor controller 284 may be configured to receive analyte sensor data from the analyte sensor 110 such as indicated with arrow 286.

The continuous analyte monitoring system 280 may further comprise a remote control 288 which is configured to receive sensor data from the sensor controller 284, such as indicated with arrow 290, and to process and/or display sensor data such as via a user interface 292. The sensor controller 284 may be configured to communicate the sensor data to the remote control 288.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### LIST OF REFERENCE NUMBERS

110 continuous analyte monitoring device
111 housing
112 analyte sensor
114 carrier
115 substrate
116 insertable portion
121 direction of insertion
122 insertion component
124 insertion cannula
126 insertion cannula holder
128 sharp end
130 slotted cannula
132 first end
134 second end
136 removable sterility cap
138 upper side
140 lower side
142 distal side
144 proximal side
146 open channel
148 direction of extension
150 upper side opening
152 lower side opening
154 electronics compartment
156 electronics unit
158 sealed compartment
160 lower housing portion
162 upper housing portion
164 connector unit
166 sterile compartment
168 opening
170 wall
172 connector unit wall
174 longitudinal side
176 first side
178 second side
180 interior space
182 interior space
184 upper connector unit side
186 lower connector unit side
188 distal connector unit side
190 proximal connector unit side
192 channel
194 first end
196 second end
198 in vivo proximal portion
200 ex vivo distal portion
202 first section
204 second section
206 electronics component
208 lower part
210 upper part
212 distal part
214 proximal part
216 upper section
218 lower section
220 base plate
222 upper plate
224 longitudinal side
226 lower surface
228 adhesive surface
230 longitudinal side
232 sensor compartment
234 sealing channel
236 input filling port
238 output filling port
240 sealing material
242 interior space
244 circumferential sealing channel
246 groove
248 surface
250 cover
252 flat surface
254 groove
256 surface
257 first arm
258 lumen
259 second arm
264 inner circumferential sealing channel
266 outer circumferential sealing channel
268 outer environment
270 wall
272 connecting channel
274 groove
276 cover
278 cavity
280 continuous analyte monitoring system
282 insertion device
284 sensor controller
286 arrow
288 remote control
290 arrow
292 user interface

What is claimed is:

1. A continuous analyte monitoring device, comprising:
an analyte sensor configured for detecting an analyte in a body fluid of a user, the analyte sensor comprising an insertable portion adapted for at least partially being inserted into the body tissue;
an insertion component comprising an insertion cannula and an insertion cannula holder, wherein the insertion cannula is attached to the insertion cannula holder and the analyte sensor is at least partially placed inside the insertion cannula;
a removable sterility cap that at least partially surrounds the insertable portion; and
a housing, comprising:
an open channel at least partially surrounding the analyte sensor and the insertion component,
an electronics compartment with an electronic unit received therein,
a lower housing portion and an upper housing portion, and
a sealing channel comprising an input filling port and an output filling port, wherein the sealing channel is at least partially filled with a sealing material configured for sealing an interior space enclosed by the upper housing portion and the lower housing portion;
wherein the removable sterility cap, the open channel and the insertion cannula holder form a sterile compartment for the insertion cannula and at least the insertable portion of the analyte sensor.

2. The continuous analyte monitoring device according to claim 1, wherein the sealing channel is configured to be filled with the sealing material in an assembled state of the lower housing portion and the upper housing portion.

3. The continuous analyte monitoring device according to claim 1, wherein the sealing material is configured for fixedly attaching the lower housing portion and the upper housing together.

4. The continuous analyte monitoring device according to claim 1, wherein the sealing channel is at least partially formed by at least one of a groove within the lower housing portion and a groove within the upper housing portion.

5. The continuous analyte monitoring device according to claim 1, wherein the housing further comprises a cavity arranged between the sealing channel and the output filling port, wherein the cavity is configured for collecting an excess of the sealing material from the sealing channel.

6. The continuous analyte monitoring device according to claim 1, wherein the housing is at least partially formed as a cylindrical ring, wherein the sealing channel is a circumferential sealing channel that seals the electronics compartment from the sterile compartment or wherein the circumferential sealing channel seals the electronics compartment from an outer environment of the continuous analyte monitoring device.

7. The continuous analyte monitoring device according to claim 1, wherein the housing further comprises a connector comprising the open channel of the housing, wherein the connector has a channel through which the analyte sensor passes and the channel connects the sterile compartment with the electronics compartment.

8. The continuous analyte monitoring device according to claim 7, wherein the channel intersects with the sealing channel.

9. The continuous analyte monitoring device according claim 8, wherein the channel and the sealing channel are at least partially filled with the sealing material configured for sealing the channel, including the analyte sensor passed through the channel, whereby the sterile compartment is sealed off from the electronics compartment.

10. The continuous analyte monitoring device according to claim 7, wherein the analyte sensor comprises an in vivo proximal portion and an ex vivo distal portion, wherein the ex vivo distal portion comprises a first section received in the channel and a second section received in the electronics compartment, wherein the first section and the second section are essentially arranged along a straight line, or wherein the first section and the second section are arranged at an angle of 20°to 80°to each other.

11. The continuous analyte monitoring device according to claim 7, wherein the lower housing portion is a lower part of the connector and wherein the upper housing portion is an upper part of the connector.

12. The continuous analyte monitoring device according to claim 7, wherein the sealing channel is divided into a first arm and a second arm by the input filling port and by the output filling port, wherein the first arm intersects with the channel, wherein at least one of: a width of a lumen of the sealing channel; a mean width of the lumen of the sealing channel; a dimension of a cross-section of the sealing channel; a mean dimension of the cross-section of the sealing channel; a cross-sectional area of the sealing channel; and a mean cross-sectional area of the sealing channel is larger in the first arm than in the second arm.

13. The continuous analyte monitoring device according to claim 1, wherein the housing comprises two of the sealing channels, wherein the two sealing channels comprise an inner circumferential sealing channel connected to an outer circumferential sealing channel via a connecting channel, and wherein the inner circumferential sealing channel has a smaller diameter than the outer circumferential sealing channel.

14. The continuous analyte monitoring device according to claim 13, wherein the housing is at least partially formed as a cylindrical ring, wherein the inner circumferential sealing channel seals the electronics compartment from the sterile compartment, wherein the outer circumferential sealing channel seals the electronics compartment from an outer environment of the continuous analyte monitoring device.

15. The continuous analyte monitoring device according to claim 1, wherein the housing comprises a base plate configured for attachment to a skin site of a user and an upper plate, wherein the base plate and the upper plate are configured for forming at least parts of longitudinal sides of the electronics compartment, wherein the lower housing portion is the base plate and wherein the upper housing portion is the upper plate.

16. A continuous analyte monitoring system, comprising:
a continuous analyte monitoring device according to claim 1;
an insertion device at least partially covering the continuous analyte monitoring device and configured for enabling a user to drive the insertion cannula into the body tissue and to insert the insertable portion of the analyte sensor;
a sensor controller coupled to the analyte sensor and configured to receive analyte sensor data from the analyte sensor; and
a remote control configured to receive sensor data from the sensor controller and to process and/or display sensor data.

17. A method of assembling the continuous analyte monitoring device according to claim 1, comprising:
a) providing the lower housing portion;
b) mounting the analyte sensor onto the lower housing portion;
c) placing the upper housing portion onto the lower housing portion;
d) injecting the sealing material into the sealing channel via the input filling port such that the lower housing portion and the upper housing portion are fixedly attached to each other and such that the interior space enclosed by the upper housing portion and the lower housing portion is sealed.

18. A method of using a continuous analyte monitoring device according to claim 1, the method comprising:
I. providing the continuous analyte monitoring device;
II. removing the removable sterility cap;
III. inserting the analyte sensor into a body tissue of the user; and
IV. removing the insertion cannula holder, thereby removing the insertion cannula from the continuous analyte monitoring device.
